(19) United States
(12) Patent Application Publication
(10) Pub. No.: US 2025/0325555 A1
(43) Pub. Date: Oct. 23, 2025

(54) Method of Treating Obesity with a Luteinizing Hormone Receptor Agonist

(71) Applicant: ICAHN SCHOOL OF MEDICINE AT MOUNT SINAI, New York, NY (US)

(72) Inventors: Daria Lizneva, New York, NY (US); Mone Zaidi, New York, NY (US); Tony Yuen, New York, NY (US)

(21) Appl. No.: 18/870,307

(22) PCT Filed: Jun. 8, 2023
(86) PCT No.: PCT/US2023/024822

§ 371 Date: Nov. 27, 2024

Related U.S. Application Data
(60) Provisional application No. 63/366,060, filed on Jun. 8, 2022.

(51) Int. Cl. A61K 31/5377; A61K 38/24; A61K 45/06; A61P 3/04

(52) U.S. Cl. CPC A61K 31/5377; A61K 38/24; A61K 45/06; A61P 3/04

(57) ABSTRACT

The present disclosure is directed to methods of treating or preventing obesity with a luteinizing hormone receptor agonist. More particularly, provided herein are methods of treating obesity in a subject in need thereof, comprising administering to the subject in need thereof a thieno[2,3-d]pyrimidine derivative.

### CLAIM OF PRIORITY

This application claims the benefit of U.S. Provisional Application Ser. No. 63/366,060, filed on Jun. 8, 2022, the contents of which are incorporated herein by reference in their entirety.

### FEDERALLY SPONSORED RESEARCH OR DEVELOPMENT

This invention was made with government support under grant number U19 AG060917, awarded by National Institute on Aging at the National Institutes of Health. The government has certain rights in the invention.

### TECHNICAL FIELD

The present disclosure is directed to a method of treating or preventing obesity with a luteinizing hormone receptor agonist. More particularly, the present disclosure is directed to a method of treating or preventing obesity with a luteinizing hormone receptor agonist, such as ORG 43553 and ORG 43902.

### BACKGROUND

Obesity is a complex disease involving an excessive amount of body fat. Obesity can increase the risk of other diseases and health problems, including heart disease, diabetes, high blood pressure and certain cancers. Obesity can occur at any age, however, advanced age can be accompanied by hormonal and lifestyle changes that increase risk of obesity. In addition, muscle mass typically decreases with age leading to a decrease in metabolism. These changes can reduce calorie needs and increase risk for obesity.

Luteinizing hormone (LH) and human chorionic gonadotropin (hCG) favor mammalian procreation and support pregnancy. Cessation of procreation coincides with the menopausal transition that is associated with an early rise in gonadotropin levels with larger changes in serum follicle-stimulating hormone (FSH) than in luteinizing hormone (LH). This transition tracks not only with rapid bone loss, but also with visceral obesity, dysregulated energy balance and reduced physical activity. In menopause, the number and amplitude of mid-cycle LH surges decrease in response to ovarian aging, while visceral fat increases from about 5-8% to about 15-20% of total body fat. Epidemiological studies also show that body mass index (BMI) is negatively associated with serum LH levels in menopausal women and women with polycystic ovary syndrome (PCOS). Therefore, there exists a need for compositions and methods for the treatment of obesity, particularly in menopausal and post-menopausal women.

### SUMMARY

Disclosed is a method of treating obesity in a subject in need thereof including administering to a subject a luteinizing hormone (LH) and human chorionic gonadotropin (hCG) receptor agonist (LH/CG receptor). The disclosure is based, at least in part, on the discovery that: (1) LH/CG receptor (LHCGR) transcripts and protein are expressed abundantly in fat tissue, with gonadal white adipose tissue levels in female mice approaching that of the ovaries themselves; (2) LHCGR activation by LH, hCG and a small molecule agonist, ORG 43553, which has undergone clinical testing for infertility, results in less fat accrual in mice on a high-fat diet independently of testosterone; and (3) ORG 43553 reduces adipocyte differentiation in organoid cultures and induces thermogenesis both in vitro and in vivo. In an embodiment, the LH/CG receptor agonist is ORG 43533 and/or ORG 43902. In an embodiment, the ORG 43533 and/or ORG 43902 is administered at a dosage of between 1 and 100 mg/kg. In an embodiment, the ORG 43533 and/or ORG 43902 is provided as a pharmaceutical composition, including a pharmaceutically acceptable carrier.

In one aspect, the disclosure provides methods of treating obesity in a subject in need thereof, including administering to the subject in need thereof a thieno[2,3-d]pyrimidine derivative according to general formula I,

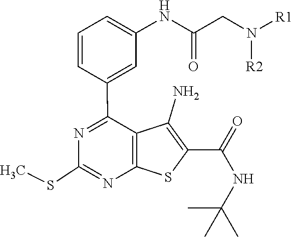

or a pharmaceutically acceptable salt thereof, wherein R1 and R2 together with the nitrogen atom to which they are bonded form a ring having 2-6 carbon atoms, optionally containing one or more heteroatoms selected from N, O and/or S. In some embodiments, the thieno[2,3-d]pyrimidine derivative is a compound selected from the group consisting of tert-butyl 5-amino-2-methylthio-4-(3-(2-(azetidin-1-yl)-acetamido)-phenyl)-thieno[2,3-d]pyrimidine-6-carboxamide; tert-butyl 5-amino-2-methylthio-4-(3-(2-(morpholin-4-yl)-acetamido)-phenyl)-thieno [2,3-d]pyrimidine-6-carboxamide; tert-butyl 5-amino-2-methylthio-4-(3-(2-(thiomorpholin-4-yl)-acetamido)-phenyl)-thieno[2,3-d]pyrimidine-6-carboxamide; tert-butyl 5-amino-2-methylthio-4-(3-(2-(piperidin-1-yl)-acetamido)-phenyl)-thieno[2,3-d]pyrimidine-6-carboxamide; tert-butyl 5-amino-2-methylthio-4-(3-(2-(pyrrolidin-1-yl)-acetamido)-phenyl)-thieno[2,3-d]pyrimidine-6-carboxamide or tert-butyl 5-amino-2-methylthio-4-(3-(2-(piperazin-1-yl)-acetamido)-phenyl)-thieno[2,3-d]pyrimidine-6-carboxamide. In some embodiments, the thieno[2,3-d]pyrimidine derivative is ORG 43533.

In some embodiments, the thieno[2,3-d]pyrimidine derivative is administered at a dosage of between 1 and 100 mg/kg. In some embodiments, the thieno[2,3-d]pyrimidine derivative is administered at a dosage of between 1 and 50 mg/kg. In some embodiments, the thieno[2,3-d]pyrimidine derivative is administered at a dosage of between 1 and 30 mg/kg. In some embodiments, the thieno[2,3-d]pyrimidine derivative is administered at a dosage of between 1 and 20 mg/kg. In some embodiments, the thieno[2,3-d]pyrimidine derivative is administered at a dosage of between 10 and 20 mg/kg. In some embodiments, the thieno[2,3-d]pyrimidine derivative is administered at a dosage of 17 mg/kg.

In some embodiments, the thieno[2,3-d]pyrimidine derivative is administered daily. In some embodiments, the thieno[2,3-d]pyrimidine derivative is administered weekly. In some embodiments, the thieno[2,3-d]pyrimidine derivative is administered for at least 26 weeks. In some embodiments, the thieno[2,3-d]pyrimidine derivative is provided as a pharmaceutical composition including a pharmaceutically acceptable carrier. In some embodiments, the methods further include administering to the subject in need thereof a luteinizing hormone (LH).

In another aspect, the disclosure provides methods of treating diet-induced obesity in a subject in need thereof, including administering to the adipose tissue of a subject in need thereof a thieno[2,3-d]pyrimidine derivative according to general formula I,

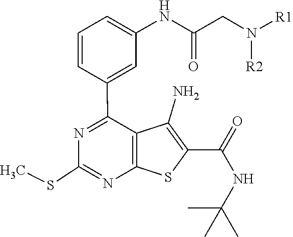

or a pharmaceutically acceptable salt thereof, wherein R1 and R2 together with the nitrogen atom to which they are bonded form a ring having 2-6 carbon atoms, optionally containing one or more heteroatoms selected from N, O and/or S. In some embodiments, the thieno[2,3-d]pyrimidine derivative is a compound selected from the group consisting of tert-butyl 5-amino-2-methylthio-4-(3-(2-(azetidin-1-yl)-acetamido)-phenyl)-thieno[2,3-d]pyrimidine-6-carboxamide; tert-butyl 5-amino-2-methylthio-4-(3-(2-(morpholin-4-yl)-acetamido)-phenyl)-thieno [2,3-d]pyrimidine-6-carboxamide; tert-butyl 5-amino-2-methylthio-4-(3-(2-(thiomorpholin-4-yl)-acetamido)-phenyl)-thieno[2,3-d]pyrimidine-6-carboxamide; tert-butyl 5-amino-2-methylthio-4-(3-(2-(piperidin-1-yl)-acetamido)-phenyl)-thieno[2,3-d]pyrimidine-6-carboxamide; tert-butyl 5-amino-2-methylthio-4-(3-(2-(pyrrolidin-1-yl)-acetamido)-phenyl)-thieno[2,3-d]pyrimidine-6-carboxamide or tert-butyl 5-amino-2-methylthio-4-(3-(2-(piperazin-1-yl)-acetamido)-phenyl)-thieno[2,3-d]pyrimidine-6-carboxamide. In some embodiments, the thieno[2,3-d]pyrimidine derivative is ORG 43902.

In some embodiments, the thieno[2,3-d]pyrimidine derivative is administered at a dosage of between 1 and 100 mg/kg. In some embodiments, the thieno[2,3-d]pyrimidine derivative is administered at a dosage of between 1 and 50 mg/kg. In some embodiments, the thieno[2,3-d]pyrimidine derivative is administered at a dosage of between 1 and 30 mg/kg. In some embodiments, the thieno[2,3-d]pyrimidine derivative is administered at a dosage of between 1 and 20 mg/kg. In some embodiments, the thieno[2,3-d]pyrimidine derivative is administered at a dosage of between 10 and 20 mg/kg. In some embodiments, the thieno[2,3-d]pyrimidine derivative is administered at a dosage of 17 mg/kg.

In some embodiments, the thieno[2,3-d]pyrimidine derivative is administered daily. In some embodiments, the thieno[2,3-d]pyrimidine derivative is administered weekly. In some embodiments, the thieno[2,3-d]pyrimidine derivative agonist is administered for at least 26 weeks. In some embodiments, the thieno[2,3-d]pyrimidine derivative is provided as a pharmaceutical composition including a pharmaceutically acceptable carrier. In some embodiments, the methods further includes administering to the subject in need thereof a luteinizing hormone (LH).

In another aspect, the disclosure provides methods of treating obesity in a subject in need thereof, comprising administering to a subject in need thereof a combination of ORG 43553 and ORG 43902.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a plot of relative Lhcgr expression as measured by qPCR in various male and female mouse fat depots.

FIG. 1B is a chromatogram of Sanger sequencing of the Lhcgr extracellular domain sequence in mouse gonadal white adipose tissue (gWAT), which was identical to the ovarian receptor.

FIG. 1C is a series of representative images of RNAscope™ in situ hybridization and immunohistochemistry (IC) showing the expression of LHCGR in various mouse fat depots and gonads in male and female C57BL/6 mice.

FIG. 1D is a plot of percent injected dose per gram tissue as measured after intravenous injection of ⁸⁹Zr-LH, in multiple organs, including mesenteric and gonadal WAT in male and female C57BL/6 mice.

FIG. 1E is a series of representative fluorescent microscopy images showing AlexaFluor-488-labeled hCG in gonads, iWAT, and gWAT of C57BL/6 and Lhcgr^{-/-} mice.

FIG. 2A is a plot of relative Lhcgr expression in mouse iWAT and in 3T3-L1 adipocytes as measured by qPCR.

FIG. 2B is a series of representative RNAscope™ in situ hybridization and immunohistochemistry (IHC) images showing LHCGR staining in differentiating 3T3-L1 murine adipocytes.

FIG. 2C is a series of representative fluorescent microscopy images showing siRNA-lentivirus-mediated knockdown of Lhcgr (Cy-3, Red) in 3T3-L1 adipocytes. DAPI, siRNA (GFP), LHCGR (CY3), and merged images are shown for siControl and siLhcgr.

FIG. 2D is a series of representative fluorescent microscopy images of specific binding of AlexaFluor-488-labeled hCG to differentiated 3T3-L1 cells.

FIG. 2E is a series of Western blots probing for ERK1/2 phosphorylation in differentiated 3T3L1 adipocytes after treatment with hCG, LH, ORG 43553.

FIG. 2F is a set of plots of log₂(fold change) for Lep, Cebpb, Cebpa, Ucp2, and Ppargc1b fat genes as measured by RNAseq in differentiated 3T3-L1 adipocytes after treatment with 1 nM LH or hCG for 12 hours.

FIG. 2G is a set of representative images and plots of oil droplet accumulation (Oil Red O staining) in differentiating 3T3-L1 adipocytes.

FIG. 3A is a plot of fat mass (g) over time (weeks) for female Lhcgr^{+/-} and Lhcgr^{-/-} mice fed normal chow compared to Lhcgr^{+/+} littermates.

FIG. 3B is a plot of lean mass (g) over time (weeks) for female Lhcgr^{+/-} and Lhcgr^{-/-} mice fed normal chow compared to Lhcgr^{+/+} littermates.

FIG. 3C is a plot of total mass (g) over time (weeks) for female Lhcgr^{+/-} and Lhcgr^{-/-} mice fed normal chow compared to Lhcgr^{+/+} littermates.

FIG. 3D is a series of representative images of H&E staining of adipocytes in iWAT and gWAT in Lhcgr^{-/-} and Lhcgr^{1/-} mice compared to Lhcgr^{1/1} littermates.

FIG. 3E is a plot of glucose tolerance among the three genotypes.

FIG. 3F is a plot of serum estrogen levels among the three genotypes.

FIG. 3G is a plot of fat mass (g) over time (weeks) for male Lhcgr^{+/-} and Lhcgr^{-/-} mice fed on a high-fat diet (HFD) compared to Lhcgr^{+/+} littermates.

FIG. 3H is a plot of lean mass (g) over time (weeks) for male Lhcgr^{+/-} and Lhcgr^{-/-} mice fed on a high-fat diet (HFD) compared to Lhcgr^{+/+} littermates.

FIG. 3I is a plot of total mass (g) over time (weeks) for male Lhcgr^{+/-} and Lhcgr^{-/-} mice fed on a high-fat diet (HFD) compared to Lhcgr^{+/+} littermates.

FIG. 3J is a series of representative images of H&E staining of adipocytes in iWAT and gWAT in Lhcgr^{-/-} and Lhcg^{+/-} mice compared to Lhcgr^{+/+} littermates.

FIG. 3K is a plot of glucose tolerance for male Lhcgr^{+/-} and Lhcgr^{-/-} mice fed on a high-fat diet (HFD) compared to Lhcgr^{+/+} littermates.

FIG. 4A is a plot of fat mass (g) of 14-week-old C57BL/6 male mice fed on high-fat diet after injection with high-dose LH or hCG injected twice-a-week.

FIG. 4B is a plot of lean mass (g) of 14-week-old C57BL/6 male mice fed on high-fat diet after injection with high-dose LH or hCG injected twice-a-week.

FIG. 4C is a plot of total mass (g) of 14-week-old C57BL/6 male mice fed on high-fat diet after injection with high-dose LH or hCG injected twice-a-week.

FIG. 4D is a plot of food intake (g) of 14-week-old C57BL/6 male mice fed on high-fat diet after injection with high-dose LH or hCG injected twice-a-week.

FIG. 4E is a plot of weight (g) of various fat depots in 14-week-old C57BL/6 male mice fed on high-fat diet after injection with high-dose LH or hCG injected twice-a-week.

FIG. 4F is a plot of glucose tolerance of 14-week-old C57BL/6 male mice fed on high-fat diet after injection with high-dose LH or hCG injected twice-a-week.

FIG. 4G is a plot of serum testosterone levels (ng/mL) of 14-week-old C57BL/6 male mice fed on high-fat diet after injection with high-dose LH or hCG injected twice-a-week.

FIG. 4H is a plot of serum leptin levels (ng/mL) of 14-week-old C57BL/6 male mice fed on high-fat diet after injection with high-dose LH or hCG injected twice-a-week.

FIG. 5A is a plot of fat mass (g) over time in mice treated with vehicle+placebo, vehicle+flutamide, hCG+placebo, or hCG+flutamide.

FIG. 5B is a plot of lean mass (g) over time in mice treated with vehicle+placebo, vehicle+flutamide, hCG+placebo, or hCG+flutamide.

FIG. 5C is a plot of total mass (g) over time in mice treated with vehicle+placebo, vehicle+flutamide, hCG+placebo, or hCG+flutamide.

FIG. 5D is a plot of food intake (g) in mice treated with vehicle+placebo, vehicle+flutamide, hCG+placebo, or hCG+flutamide.

FIG. 5E is a plot of weight (g) of various fat depots in mice treated with vehicle+placebo, vehicle+flutamide, hCG+placebo, or hCG+flutamide.

FIG. 5F is a plot of glucose tolerance in mice treated with vehicle+placebo, vehicle+flutamide, hCG+placebo, or hCG+flutamide.

FIG. 5G is a is a plot of leptin levels (ng/ml) in mice treated with vehicle+placebo, vehicle+flutamide, hCG+placebo, or hCG+flutamide.

FIG. 6A is a plot of fat mass (g) of 14-week-old C57BL/6 mice (25 mice/group) treated with subcutaneous injections of vehicle or ORG 43553 (17 mg/kg), and fed on high-fat diet for 9 weeks.

FIG. 6B is a plot of total mass (g) of 14-week-old C57BL/6 mice (25 mice/group) treated with subcutaneous injections of vehicle or ORG 43553 (17 mg/kg), and fed on high-fat diet for 9 weeks.

FIG. 6C is a plot of lean mass (g) of 14-week-old C57BL/6 mice (25 mice/group) treated with subcutaneous injections of vehicle or ORG 43553 (17 mg/kg), and fed on high-fat diet for 9 weeks.

FIG. 6D is a plot of food intake (g) of 14-week-old C57BL/6 mice (25 mice/group) treated with subcutaneous injections of vehicle or ORG 43553 (17 mg/kg), and fed on high-fat diet for 9 weeks.

FIG. 6E is a plot of weight (g) of various fat depots of 14-week-old C57BL/6 mice (25 mice/group) treated with subcutaneous injections of vehicle or ORG 43553 (17 mg/kg), and fed on high-fat diet for 9 weeks.

FIG. 6F is a plot of glucose tolerance of 14-week-old C57BL/6 mice (25 mice/group) treated with subcutaneous injections of vehicle or ORG 43553 (17 mg/kg), and fed on high-fat diet for 9 weeks.

FIG. 6G is a plot of serum testosterone levels (ng/mL) of 14-week-old C57BL/6 mice (25 mice/group) treated with subcutaneous injections of vehicle or ORG 43553 (17 mg/kg), and fed on high-fat diet for 9 weeks.

FIG. 6H is a plot of serum leptin levels (ng/mL) of 14-week-old C57BL/6 mice (25 mice/group) treated with subcutaneous injections of vehicle or ORG 43553 (17 mg/kg), and fed on high-fat diet for 9 weeks.

FIG. 7A is a plot of relative Lhcgr expression levels as measured by qPCR in white adipose tissue (WAT) depots in male and female C57BL/6 mice.

FIG. 7B is a series of representative images of RNAscope™ in situ hybridization showing the expression of Lhcgr in various mouse fat depots and gonads in male and female Lhcgr^{-/-} and Lhcgr^{+/+} mice.

FIG. 7C is a the polynucleotide and amino acid sequence of the LHCGR ectodomain (shaded) validated by Sanger sequencing, which was identical to the ovarian receptor

FIG. 7D is a series of representative immunohistochemistry images showing the expression of LHCGR in various mouse fat depots and gonads in male and female Lhcgr^{-/-} and Lhcgr^{+/+} mice.

FIG. 7E is a series of representative fluorescent microscopy images of intraperitoneal AlexaFluor-488-labeled hCG bound to gonadal and subcutaneous fatpads in male and female Lhcgr^{-/-} and Lhcgr^{-/+} mice.

FIG. 7F is a plot of percent injected dose per gram tissue as measured after intravenous injection of ⁸⁹Zr-LH, in multiple organs, including mesenteric and gonadal WAT in male and female mice.

FIG. 7G is a is a series of Western blots probing for ERK1/2 phosphorylation in differentiated 3T3L1 adipocytes after treatment with hCG, LH, ORG 43553.

FIG. 8A is a plot of fat mass (g) of female Lhcgr^{+/-} and Lhcgr^{+/+} mice on normal chow over time and a plot of serum estrogen levels over the same time interval.

FIG. 8B is a plot of fat mass (g) of male Lhcgr^{+/-} and Lhcgr^{+/+} mice on high-fat diet over time and a plot of serum testosterone levels over the same time interval.

FIG. 8C is a plot of fat mass (g) and food intake (g) of 14-week-old male C57BL/6 mice (N=20/group) fed on high-fat diet and injected intraperitoneally with LH (250 ng), hCG (250 ng) or vehicle (200 μL saline), twice-a-week, for 6 weeks.

FIG. 8D is a plot of fat mass (g) in male Lhcgr^{-/-} mice fed on high-fat diet and injected intraperitoneally with LH (250 ng) or vehicle (200 μL saline), twice-a-week, for 6 weeks.

FIG. 8E is a plot of fat mass (g) and food intake (g) of 14-week-old male C57BL/6 mice (N=20/group) fed on high-fat diet and injected intraperitoneally with vehicle, vehicle and flutamide, hCG, or hCG and flutamide twice a week for three weeks.

FIG. 8F is a plot (left) of fat mass (g) and food intake (g) of 14-week-old male C57BL/6 mice injected with LHCGR agonist ORG 43553 (17 mg/kg, subcutaneous, twice-a-week), or vehicle for 9 weeks. At center is a plot of tissue weight (g) of various fat depots of 14-week-old male C57BL/6 mice injected with LHCGR agonist ORG 43553 (17 mg/kg, subcutaneous, twice-a-week), or vehicle for 9 weeks. At right is a plot of serum testosterone levels (ng/mL) for the same mice.

FIG. 8G is a plot of distribution of adipocytes in iWAT of 14-week-old male C57BL/6 mice treated with ORG 43553 (17 mg/kg, subcutaneous, twice-a-week) or vehicle for 9 weeks. Also shown is the total adipocyte area of cell size ≤4000 μm² and cell size ≤4000 μm², as well as representative images of H&E staining of the adipocytes.

FIG. 8H is a plot of fat mass (g) (top), total mass (g) (middle), tissue weight (g) (bottom left), and testosterone levels (ng/mL) (bottom right) of 14-week-old male C57BL/6 mice injected with ORG 43553 (17 mg/kg, subcutaneous, twice-a-week) or vehicle for 9 weeks.

FIG. 9A is a series of representative microscopy images of Oil Red O staining of 3T3.L1 adipocytes treated with ORG 43553, hCG, or vehicle, and plots of quantification of Oil Red O staining.

FIG. 9B is a series of representative images of paraffin sections of organoids derived from 3T3-L1 cells. Images of H&E staining, RNAscope™ in situ hybridization, and immunohistochemistry are shown for organoids treated with vehicle, LH, hCG, and ORG 43553. Also shown is the quantitation of the thickness of the fat cell layer (μm).

FIG. 9C is a plot (left) of oxygen consumption rate (OCR) for 3T3-L1 adipocytes that were pretreated with ORG 43553 for 1 hour before measuring OCR on a Seahorse Xf96 Analyzer. At right is a plot of OCR for 3T3-L1 adipocytes after treatment with ORG 43553, LH, or hCG at basal measurements and after addition of oligomycin.

FIG. 9D is a plot of oxygen consumption (VO₂) in ORG 43553-treated mice on high-fat diet. At right is a table of VO₂ and energy expenditure for mice treated with ORG 43553 or vehicle.

FIG. 10A is the chemical structure of ORG 43553 [5-amino-2-methylsulfanyl-4-[3-(2-morpholin-4-yl-acetylamino)-phenyl]-thieno[2,3-d]pyrimidine-6-carboxylic acid tert-butylamide].

FIG. 10B is a schematic of the binding of ORG 43553 with mouse LHCGR.

FIG. 10C is a schematic of side (left) and top (right) views of ORG 43553 in complex with LHCGR on 280 nanosecond molecular dynamics

### DETAILED DESCRIPTION

Luteinizing Hormone (LH) and Human Chorionic Gonadotropin (hCG) Receptor

The luteinizing hormone/choriogonadotropin receptor (LHCGR) is a member of a subfamily of G protein-coupled receptors (GPCR) characterized by the presence of a large N-terminal extracellular domain containing several leucine-rich repeats (LRR). This glycoprotein hormone receptor family has been named the LRR-containing GPCR (LGR) family. The human LHCGR protein is encoded by the LHCGR gene at cytogenetic location 2p16.3, genomic coordinates (GRCh38): 2:48,686,774-48,755,724. The amino acid sequence of the human LHCGR precursor protein is provided below as SEQ ID NO: 1.

(SEQ ID NO: 1)
MKQRFSALQLLKLLLLLQPPLPRALREALCPEPCNCVPDGALRCPGPTAG
LTRLSLAYLPVKVIPSQAFRGLNEVIKIEISQIDSLERIEANAFDNLLNL
SEILIQNTKNLRYIEPGAFINLPRLKYLSICNTGIRKFPDVTKVFSSESN
FILEICDNLHITTIPGNAFQGMNNESVTLKLYGNGFEEVQSHAFNGTTLT
SLELKENVHLEKMHNGAFRGATGPKTLDISSTKLQALPSYGLESIQRLIA
TSSYSLKKLPSRETFVNLLEATLTYPSHCCAFRNLPTKEQNFSHSISENF
SKQCESTVRKVNNKTLYSSMLAESELSGWDYEYGFCLPKTPRCAPEPDAF
NPCEDIMGYDFLRVLIWLINILAIMGNMTVLFVLLTSRYKLTVPRFLMCN
LSFADFCMGLYLLLIASVDSQTKGQYYNHAIDWQTGSGCSTAGFFTVFAS
ELSVYTLTVITLERWHTITYAIHLDQKLRLRHAILIMLGGWLFSSLIAML
PLVGVSNYMKVSICFPMDVETTLSQVYILTILILNVVAFFIICACYIKIY
FAVRNPELMATNKDTKIAKKMAILIFTDFTCMAPISFFAISAAFKVPLIT
VTNSKVLLVLFYPINSCANPFLYAIFTKTFQRDFFLLLSKFGCCKRRAEL
YRRKDFSAYTSNCKNGFTGSNKPSQSTLKLSTLHCQGTALLDKTRYTEC

In some embodiments, the methods disclosed herein include administering to a subject one or more LHCGR agonists. In some embodiments, the LHCGR agonist is a thieno[2,3-d]pyrimidine derivative. In some embodiments, the thieno[2,3-d]pyrimidine derivative is a compound according to general formula I,

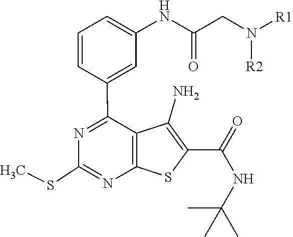

or a pharmaceutically acceptable salt thereof. In some embodiments, R1 and R2 together with the nitrogen atom to which they are bonded form a ring having 2-6 carbon atoms, optionally containing one or more heteroatoms selected from N, O and/or S. In some embodiments, the thieno[2,3-d]pyrimidine derivative is a compound selected from the group consisting of tert-butyl 5-amino-2-methylthio-4-(3-(2-(azetidin-1-yl)-acetamido)-phenyl)-thieno[2,3-d]pyrimidine-6-carboxamide; tert-butyl 5-amino-2-methylthio-4-(3-(2-(morpholin-4-yl)-acetamido)-phenyl)-thieno [2,3-d]pyrimidine-6-carboxamide; tert-butyl 5-amino-2-methylthio-4-(3-(2-(thiomorpholin-4-yl)-acetamido)-phenyl)-thieno[2,3-d]pyrimidine-6-carboxamide; tert-butyl 5-amino-2-methylthio-4-(3-(2-(piperidin-1-yl)-acetamido)-phenyl)-thieno[2,3-d]pyrimidine-6-carboxamide; tert-butyl 5-amino-2-methylthio-4-(3-(2-(pyrrolidin-1-yl)-acetamido)-phenyl)-thieno[2,3-d]pyrimidine-6-carboxamide or tert-butyl 5-amino-2-methylthio-4-(3-(2-(piperazin-1-yl)-acetamido)-phenyl)-thieno[2,3-d]pyrimidine-6-carboxamide.

In some embodiments, the thieno[2,3-d]pyrimidine derivative is ORG 43533. ORG 43553 is an orally active and low molecular weight (LMW) LHCGR agonist. ORG 43553 shows agonistic activity to human LHCGR with an EC₅₀ value of 3.7 nM. The structure of ORG 43553 is provided below:

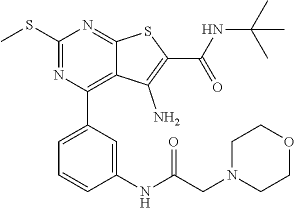 [SMILES: CSc1nc(-c2cccc(NC(=O)CN3CCOCC3)c2)c2c(N)c(C(=O)NC(C)(C)C)sc2n1]

### Pharmaceutical Compositions

In some aspects, the compositions and methods described herein include the manufacture and use of pharmaceutical compositions and medicaments that include one or more compounds as disclosed herein. Also included are the pharmaceutical compositions themselves.

In some aspects, the compositions disclosed herein can include other compounds, drugs, or agents used for the treatment. For example, in some instances, pharmaceutical compositions disclosed herein can be combined with one or more (e.g., one, two, three, four, five, or less than ten) compounds.

In some aspects, the pH of the compositions disclosed herein can be adjusted with pharmaceutically acceptable acids, bases, or buffers to enhance the stability of the compounds or its delivery form.

Pharmaceutical compositions typically include a pharmaceutically acceptable carrier, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable carrier, adjuvant, or vehicle is a composition that can be administered to a patient, together with a compound of the invention, and which does not destroy the pharmacological activity thereof and is nontoxic when administered in doses sufficient to deliver a therapeutic amount of the compound. Exemplary conventional nontoxic pharmaceutically acceptable carriers, adjuvants, and vehicles include saline, solvents, dispersion media, coatings, antibacterial and antifungal agents, isotonic and absorption delaying agents, and the like, compatible with pharmaceutical administration.

In particular, pharmaceutically acceptable carriers, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

As used herein, the compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivative thereof.

In particular, pharmaceutically acceptable salts of the compounds disclosed herein include, e.g., those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include, e.g., alkali metal (e.g., sodium), alkaline earth metal (e.g., magnesium), ammonium salts. The invention also envisions the quaternization of any basic nitrogen-containing groups of the compounds disclosed herein. Water or oil-soluble or dispersible products can be obtained by such quaternization.

In some aspects, the pharmaceutical compositions disclosed herein can include an effective amount of one or more compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome. In some aspects, pharmaceutical compositions can further include one or more additional compounds, drugs, or agents used for the treatment in amounts effective for causing an intended effect or physiological outcome.

In some aspects, the pharmaceutical compositions disclosed herein can be formulated for sale in the United States, import into the United States, or export from the United States.

The present disclosure also encompasses the therapeutic combinations disclosed herein in the form of a kit or packaged formulation. A kit or packaged formulation as used herein includes one or more dosages of a subject peptide, and salts thereof, in a container holding the dosages together with instructions for simultaneous or sequential administration to a patient. For example, the package may contain the peptides along with a pharmaceutical carrier combined in the form of a powder for mixing in an aqueous solution, which can be ingested by the afflicted subject. The package or kit includes appropriate instructions, which encompasses diagrams, recordings (e.g., audio, video, compact disc), and computer programs providing directions for use of the combination therapy. The foregoing descriptions of specific embodiments of the present disclosure have been presented for purposes of illustration and description. They are not intended to be exhaustive or to limit the disclosure to the precise forms disclosed, and obviously many modifications and variations are possible in light of the above teaching.

### Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of a compound of the formulae described herein and one or more additional compounds (e.g., one or more additional compounds, drugs, or agents used for the treatment of obesity or any other obesity-related condition or disease, including conditions or diseases known to be associated with or caused by obesity), both the compound and the additional compound should be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

### Combination Therapy

In various embodiments, a compound described herein or composition described herein may be administered to a subject in combination with one or more other therapies (e.g., biologic or antibody therapies). In some embodiments, a pharmaceutical composition described herein may be administered to a subject in combination with one or more therapies. The one or more other therapies may be in the same composition or a different composition as a compound described herein.

In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In some embodiments, two or more therapies are administered concurrently. The two or more therapies can be administered in the same composition or a different composition. Further, the two or more therapies can be administered by the same route of administration of a different route of administration.

### Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, the present disclosure provides methods for using a composition comprising a compound, including pharmaceutical compositions (indicated below as ‘X’) disclosed herein in the following methods:

Substance X for use as a medicament in the treatment of one or more diseases or conditions disclosed herein. Use of substance X for the manufacture of a medicament for the treatment of Y; and substance X for use in the treatment of Y.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of obesity or obesity-related diseases.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition from which the subject is suffering. In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder are ameliorated or otherwise beneficially altered. As used herein, amelioration of the symptoms of a particular disorder refers to any lessening, whether permanent or temporary, lasting or transient that can be attributed to or associated with treatment by the compositions and methods of the present invention.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention.

As used herein, the term “preventing a disease” in a subject means for example, to stop the development of one or more symptoms of a disease in a subject before they occur or are detectable, e.g., by the patient or the patient's doctor. Preferably, the disease does not develop at all, i.e., no symptoms of the disease are detectable. However, it can also mean delaying or slowing of the development of one or more symptoms of the disease. Alternatively, or in addition, it can mean decreasing the severity of one or more subsequently developed symptoms.

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

Publications disclosed herein are provided solely for their disclosure prior to the filing date of the present disclosure. Nothing herein is to be construed as an admission that the present disclosure is not entitled to antedate such publication. Further, the dates of publication provided may be different from the actual publication dates which may need to be independently confirmed.

Each of the applications and patents cited in this text, as well as each document or reference, patent or non-patent literature, cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself, and, each of these documents or references (“herein-cited references”), as well as each document or reference cited in each of the herein-cited references (including any manufacturer's specifications, instructions, etc.), is hereby expressly incorporated herein by reference.

The following non-limiting examples serve to further illustrate the present disclosure.

### EXAMPLES

The invention is further described in the following examples, which do not limit the scope of the invention described in the claims.

### Methods

The following materials and methods were used in the following examples.

qPCR: Total RNA was isolated using TRIzol reagent (Invitrogen, 15596018) and reverse transcribed (Invitrogen, 18064014). qPCR has been performed using SYBR Green Master Mix (Applied Biosystems 4472918).

SANGER SEQUENCING: RNA from mouse genital adipose tissue was reverse transcribed (Invitrogen 18080093). A fragment of Lhcgr was amplified by PCR and sequenced.

RNASCOPE™: Paraffin sections were processed and stained using the RNAscope™ Probe-Mm-Lhcgr (ACD 408171) and RNAscope™ 2.5HD Reagent Kit-Brown (ACD 322300). IHC: Paraffin sections were stained with rabbit LHCGR polyclonal antibodies (Invitrogen, PA5-79598) and anti-rabbit HRP-IgG (Leica RE7161).

BIODISTRIBUTION: Male and female C57BL/6 mice (5 mice/group) were injected i.v. with ⁸⁹Zr-LH, individual tissues were dissected 2 hours after for γ-counting.

hCG BINDING: Recombinant hCG was conjugated with Alexa Fluor 488 Microscale Protein Labelling Kit (Invitrogen A30006) and injected i.p. (0.45 μg).

CELL CULTURE: Differentiated 3T3-L1 preadipocytes (Zen-Bio) were treated with hCG or LH for 20 minutes.

siRNA KNOCKDOWN: Lentiviral vectors expressing Lhcgr siRNA (ABM 2649609) or siControl (ABM LVP015-G) were used per manufacturer's recommendation.

WESTERN BLOTTING: Proteins were blotted with commercial antibodies (Cell Signaling, Phospho-ERK1/2, 9101S; ERK1/2, 4695S, 1:5000).

STATISTICS: Two-tailed Student's t test or two-way ANOVA were used (*p<0.05; **p<0.01).

### Example 1: Lhcgr Expression, Binding and Activation in Mouse Adipocytes

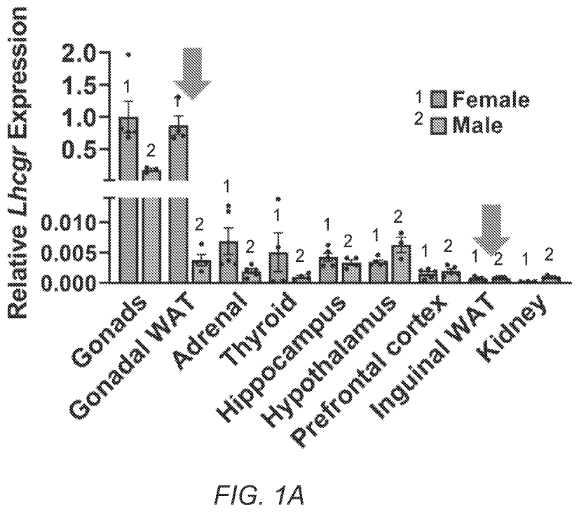

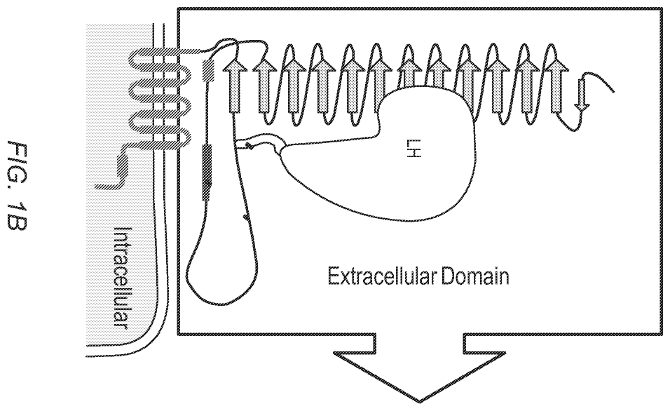

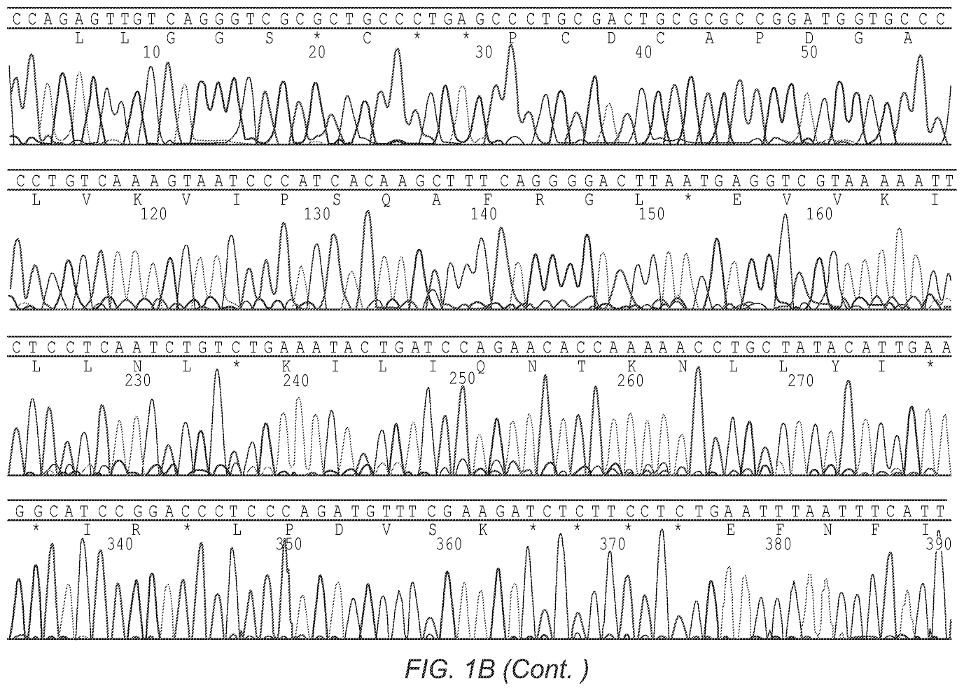

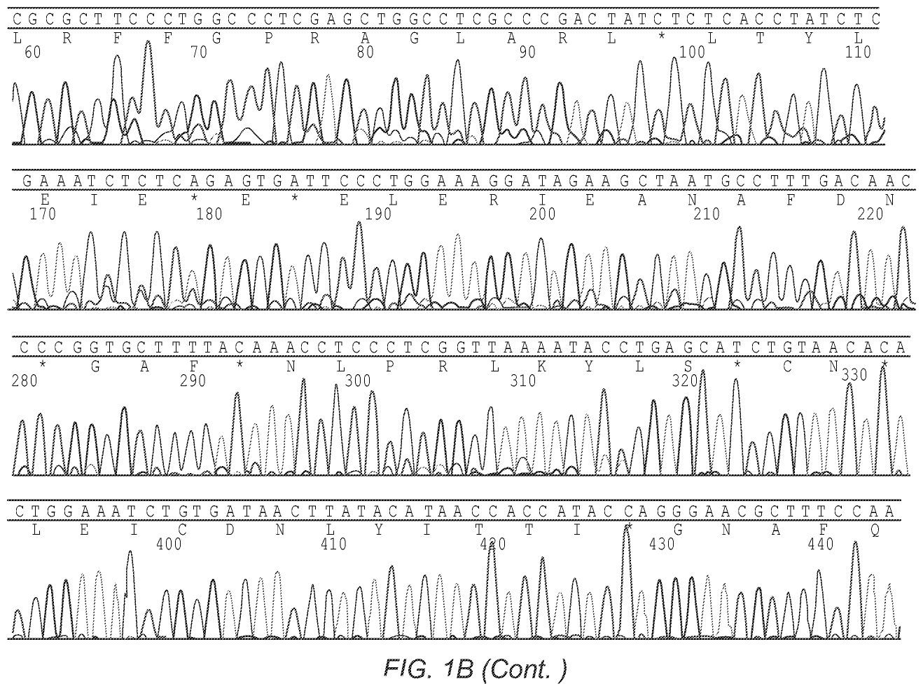

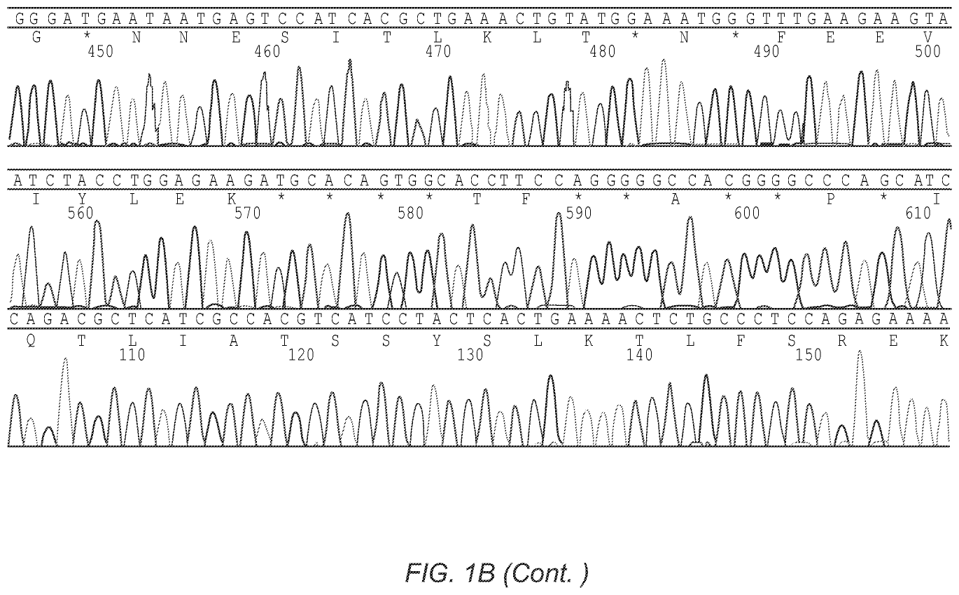

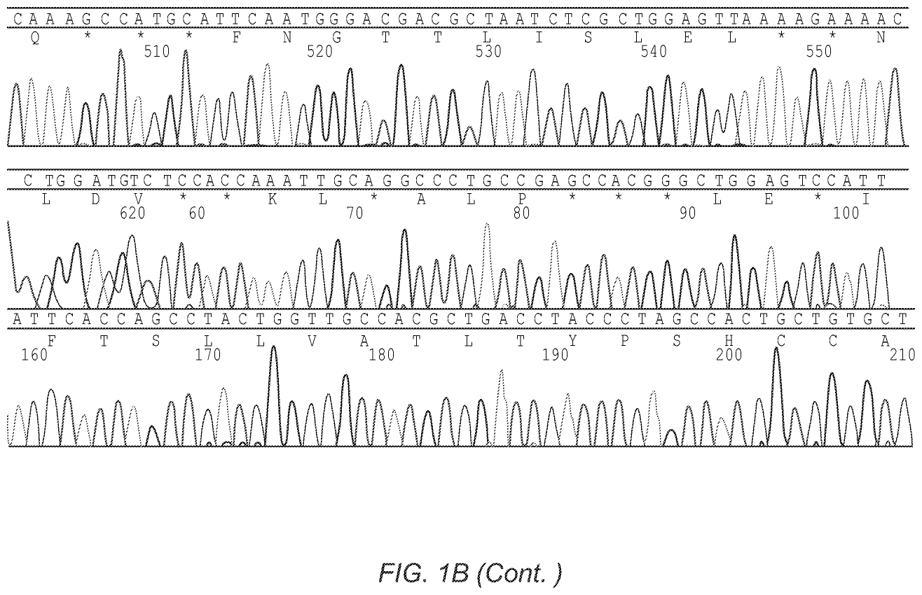

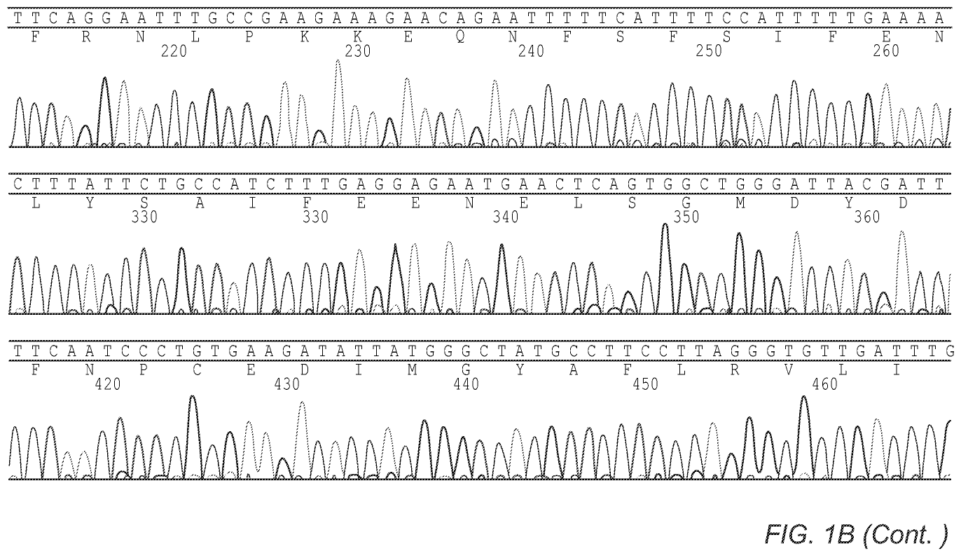

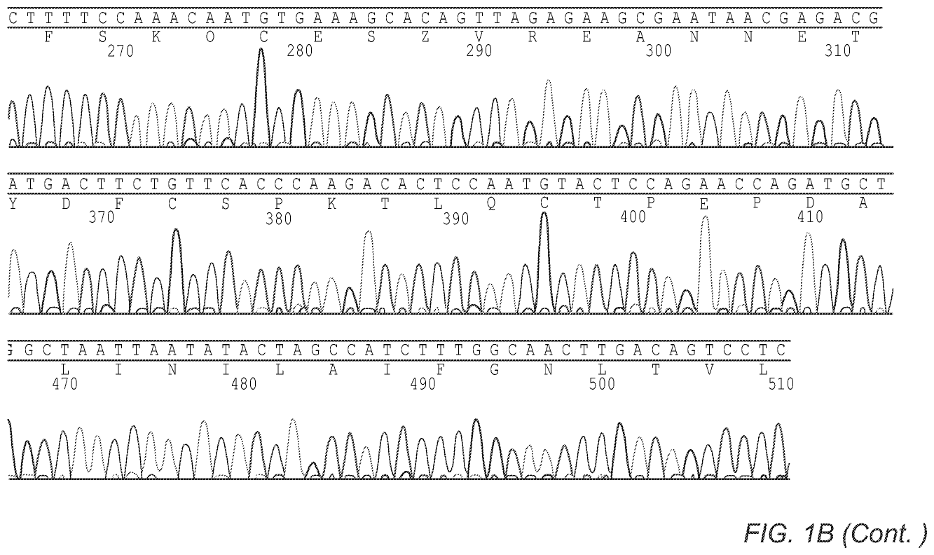

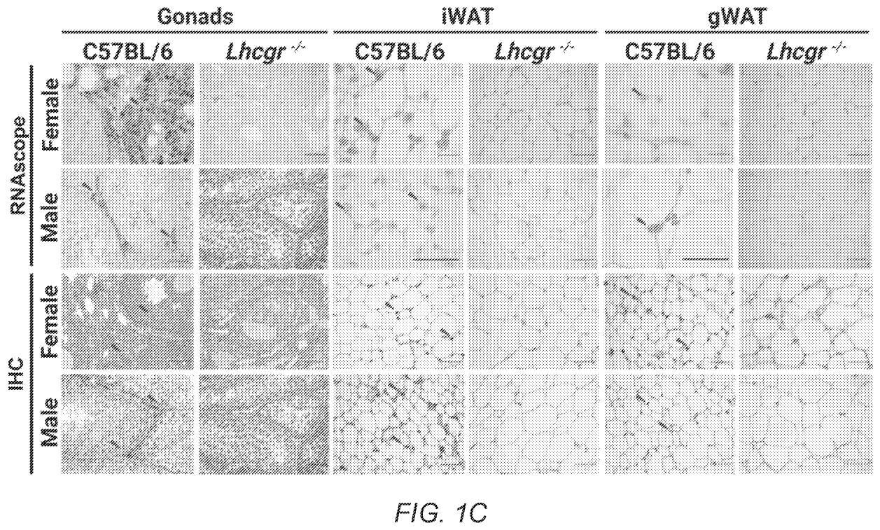

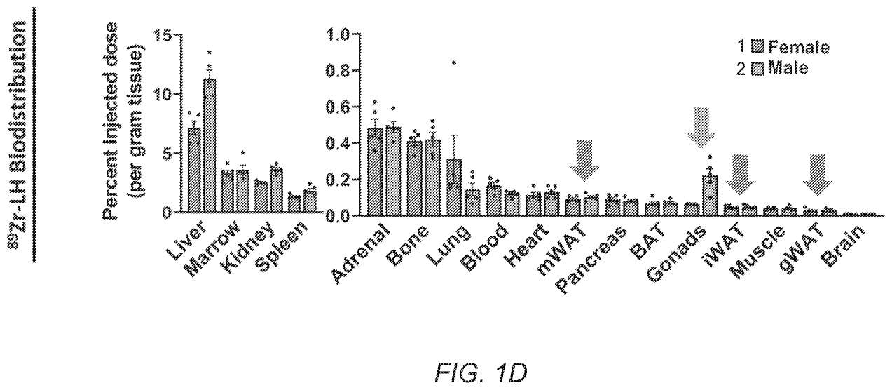

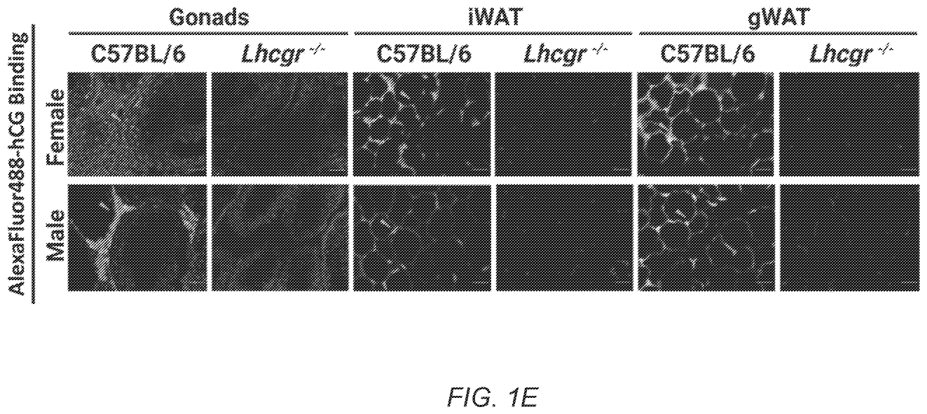

qPCR was used to measure relative expression in various male and female mouse fat depots, with results plotted in FIG. 1A. As shown in FIG. 1B, Lhcgr extracellular domain sequence in mouse gonadal white adipose tissue (gWAT) was identical to the sequence in the ovarian receptor. As shown in FIG. 1C, RNAscope™ in situ hybridization and immunohistochemistry (IHC) were used to visualize the expression of LHCGR in various mouse fat depots and gonads in male and female C57BL/6 mice. No staining was detected in Lhcgr^{-/-} mice, indicating specificity of the LHCGR probe and antibody. As shown in FIG. 1D, significant concentrations of ⁸⁹Zr-LH, injected intravenously, were detected in multiple organs, including mesenteric and gonadal WAT. As shown in FIG. 1E, AlexaFluor-488-labeled hCG, injected intraperitoneally, was localized in mouse iWAT and gWAT. This hCG binding further confirms the presence of LHCGR in adipose tissue.

### Example 2: LHCGR is Expressed in Adipocytes and is Fully Functional

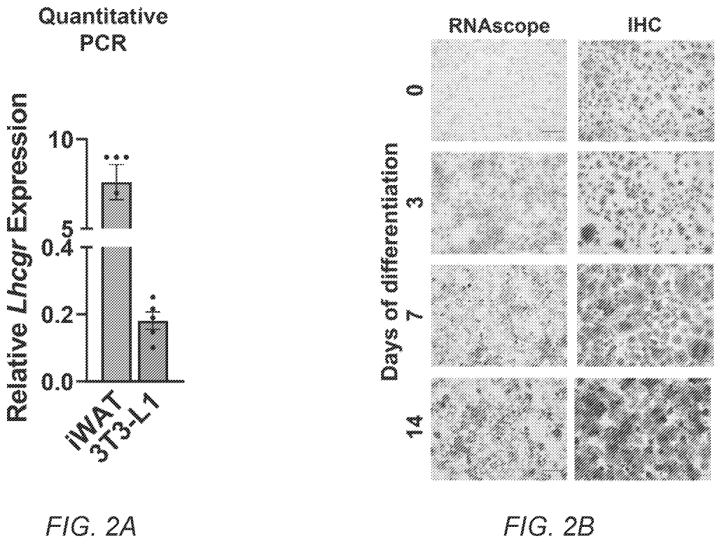

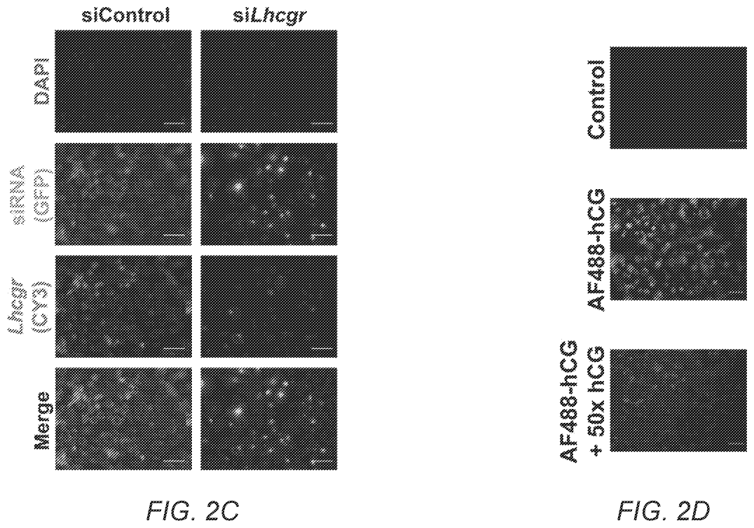

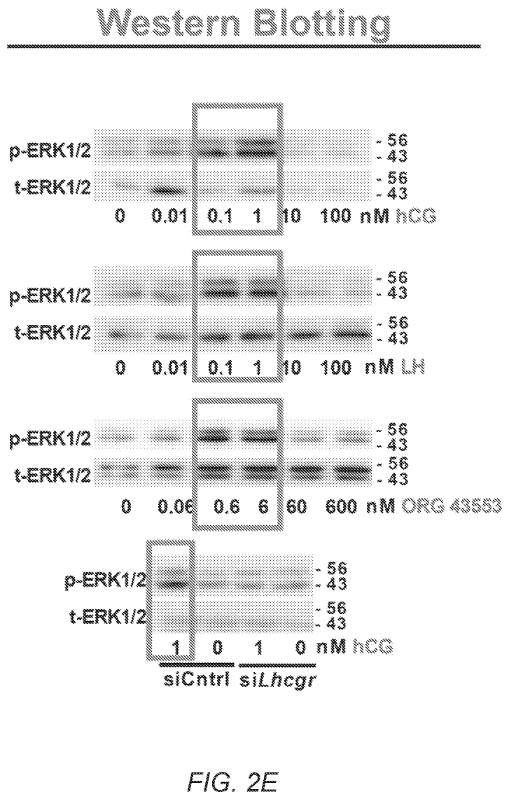

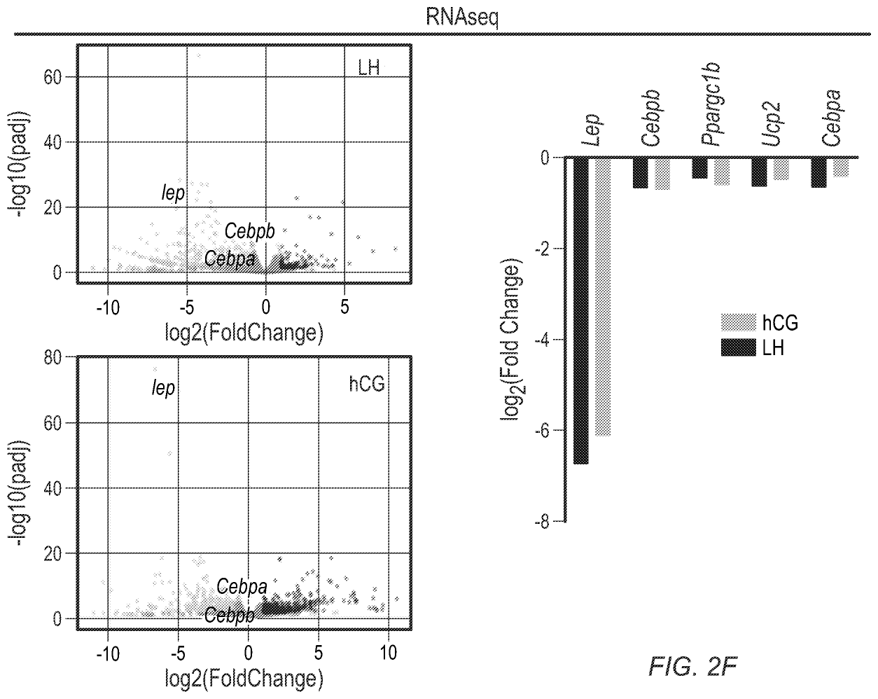

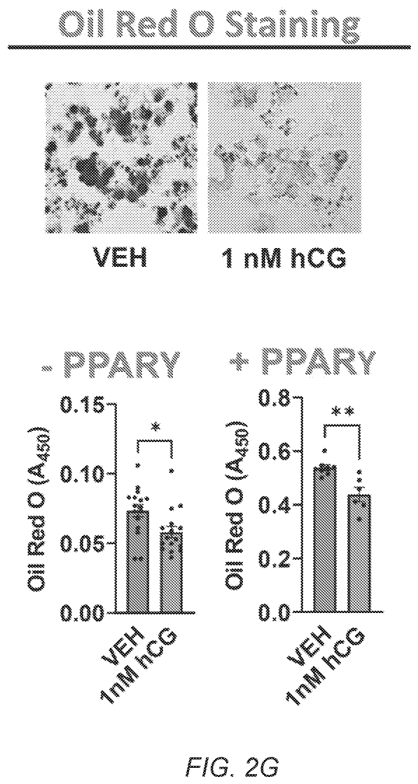

As shown in FIG. 2A, relative Lhcgr expression in mouse iWAT and in 3T3-L1 adipocytes as measured by qPCR indicates that Lhcgr is expressed in mouse iWAT and in 3T3-L1 adipocytes. As shown in FIG. 2B, RNAscope™ in situ hybridization and immunohistochemistry (IHC) indicates LHCGR staining in differentiating 3T3-L1 murine adipocytes. As shown in FIG. 2C, siRNA-lentivirus-mediated knockdown of Lhcgr (Cy-3, Red) in 3T3-L1 adipocytes confirmed antibody specificity. The GFP reporter (Green) showed high efficiency of lentivirus infection. As shown in FIG. 2D, binding of AlexaFluor-488-labeled hCG to differentiated 3T3-L1 cells was specific, as this binding could be competitively displaced by pretreatment with unlabeled hCG. As shown in FIG. 2E, LH and hCG induced ERK1/2 phosphorylation in differentiated 3T3L1 adipocytes in a dose-dependent manner, suggesting that the adipose LHCGR is fully functional. The hCG-induced ERK1/2 phosphorylation was abrogated with LHCGR siRNA knockdown, further suggesting that ERK1/2 phosphorylation by LH acts through LHCGR in adipocytes. RNAseq was performed to measure the log₂(fold change) for Lep, Cebpb, Cebpa, Ucp2, and Ppargc1b fat genes in differentiated 3T3-L1 adipocytes after treatment with 1 nM LH or hCG for 12 hours. As shown in FIG. 2F, some fat genes were downregulated, including Leptin (Lep). As shown in FIG. 2G, treatment with hCG reduced oil droplet accumulation (Oil Red O staining) in differentiating 3T3-L1 adipocytes.

### Example 3: Lhcgr Deficiency Provokes Weight Gain in Female Mice on Normal Chow and in Male Mice on High-Fat Diet (HFD)

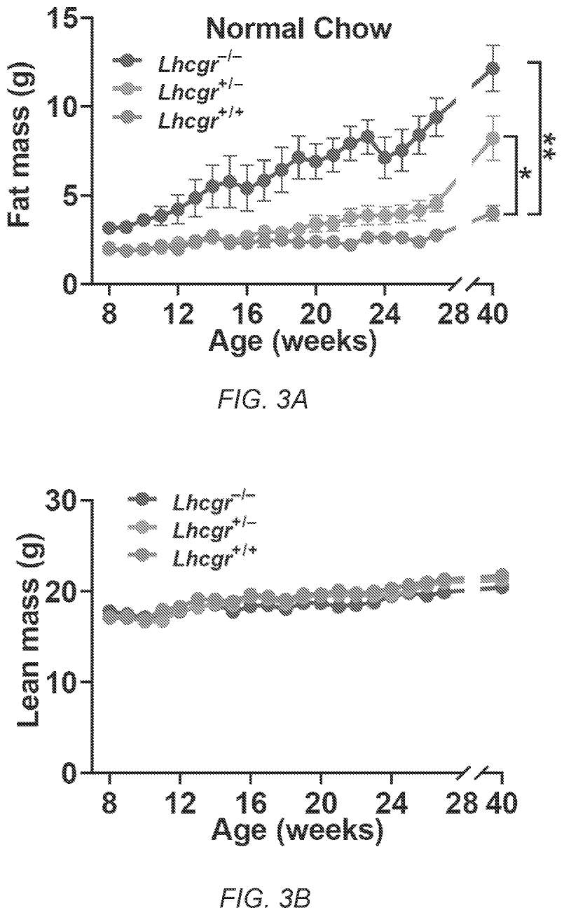

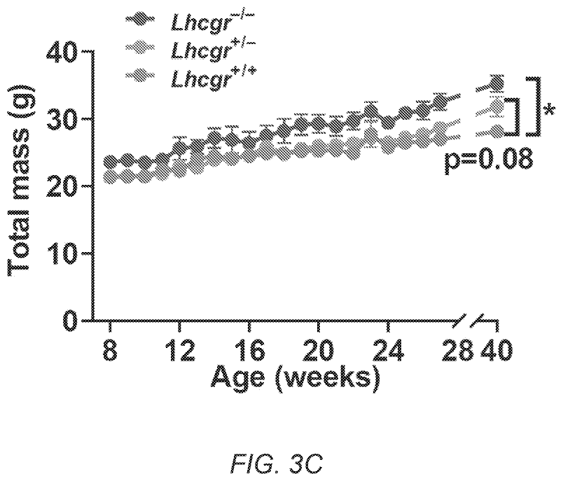

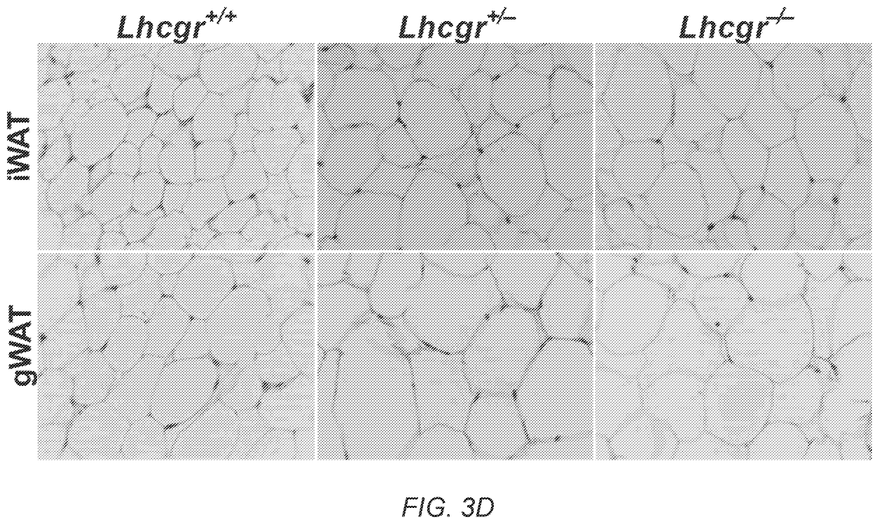

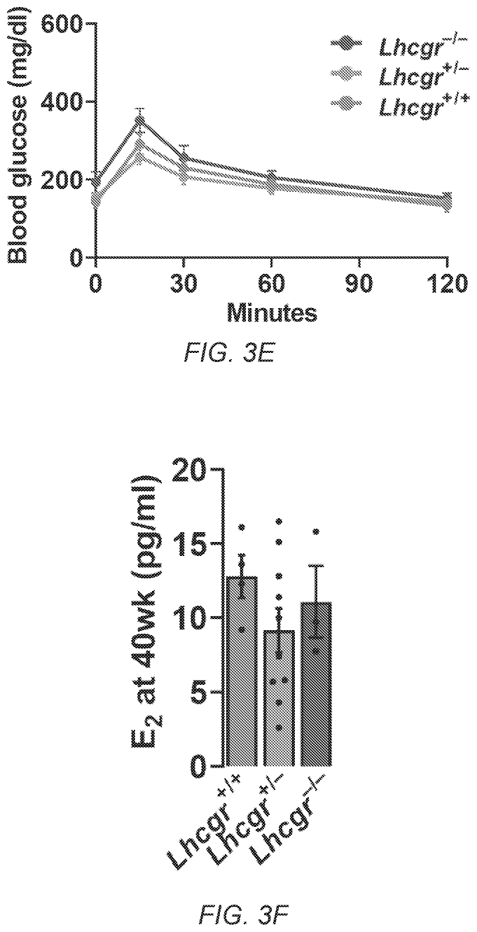

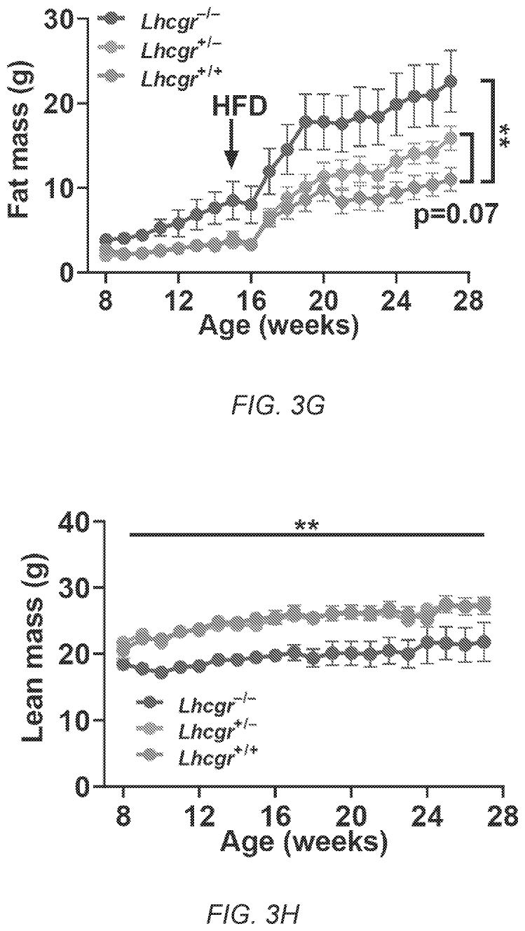

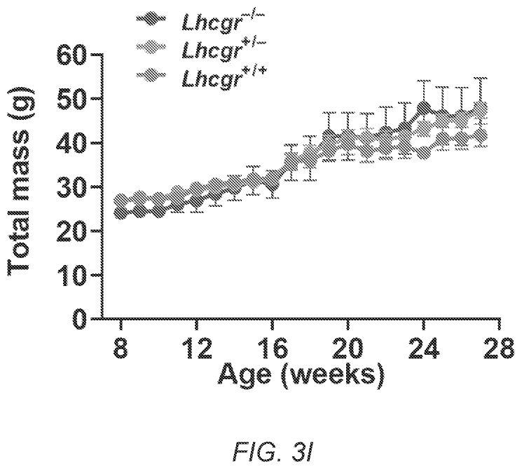

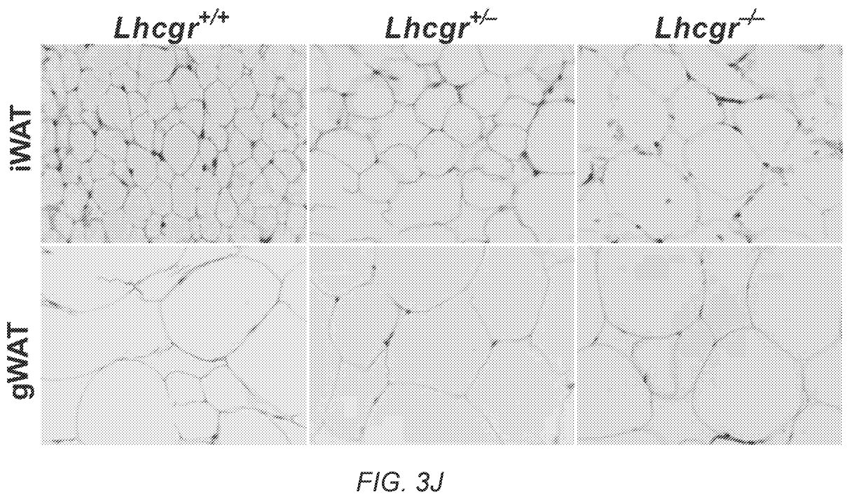

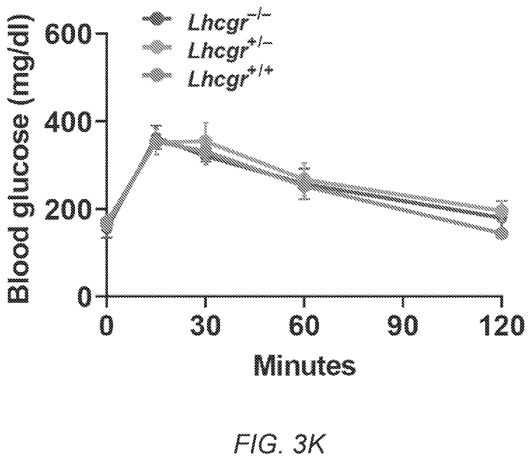

Male and female Lhcgr^{+/+}, Lhcgr^{+/-} and Lhcgr^{-/-} mice were fed with normal chow or high-fat diet for 40 weeks. As shown in FIG. 3A, female Lhcgr^{+/-} and Lhcgr^{-/-} mice fed normal chow displayed higher fat mass compared to Lhcgr^{+/+} littermates, indicating the anti-adiposity action of LH. As shown in FIG. 3C, Lhcgr^{-/-} mice also displayed higher total mass compared to Lhcgr^{+/+} mice. As shown in FIG. 3D, H&E staining indicated larger adipocytes in iWAT and gWAT in Lhcgr^{-/-} and Lhcgr^{-/-} mice relative to Lhcgr^{+/+} littermates. As shown in FIGS. 3B, 3E, and 3F, there was no difference in the lean mass, glucose tolerance, or serum estrogen among the three genotypes. As shown in FIG. 3G, on a high-fat diet, male Lhcgr^{+/-} and Lhcgr^{-/-} mice displayed higher fat mass compared to Lhcgr^{+/+} mice. As shown in FIG. 3I, there was no difference in total mass in the three genotypes. As shown in FIG. 3J, H&E staining indicated larger adipocytes in iWAT and gWAT in male Lhcgr^{-/-} and Lhcgr^{+/-} mice on a high-fat diet. As shown in FIG. 3H, male Lhcgr^{-/-} mice on HFD displayed lower lean mass compared to Lhcgr^{+/+} and Lhcgr^{+/-} littermates. As shown in FIG. 3K, glucose tolerance was the same among different genotypes.

### Example 4: LH and hCG Reduce HFD-Induced Weight Gain in C57BL/6 Mice

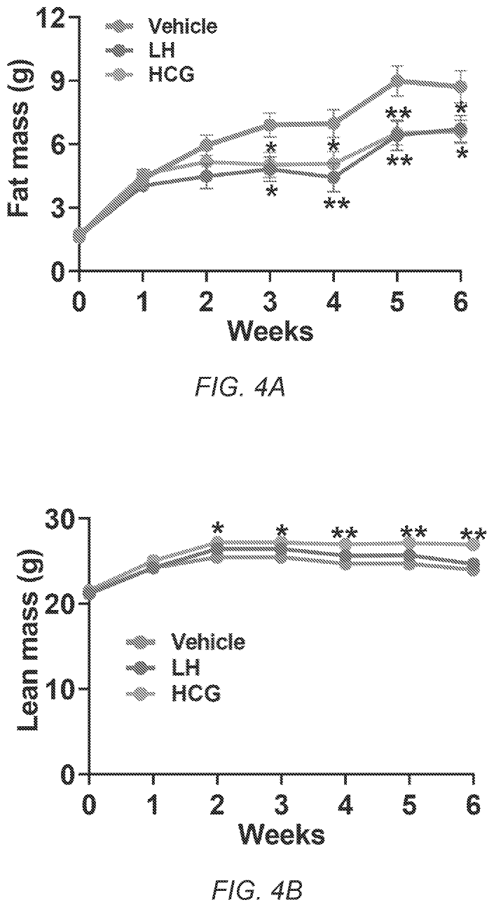

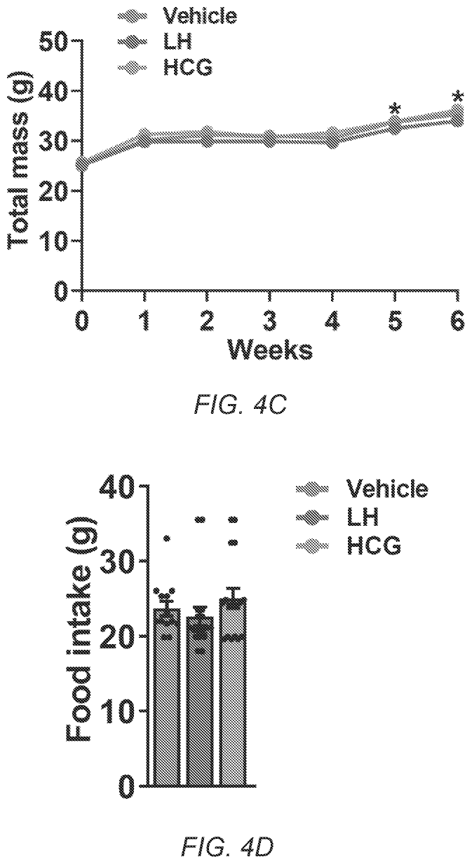

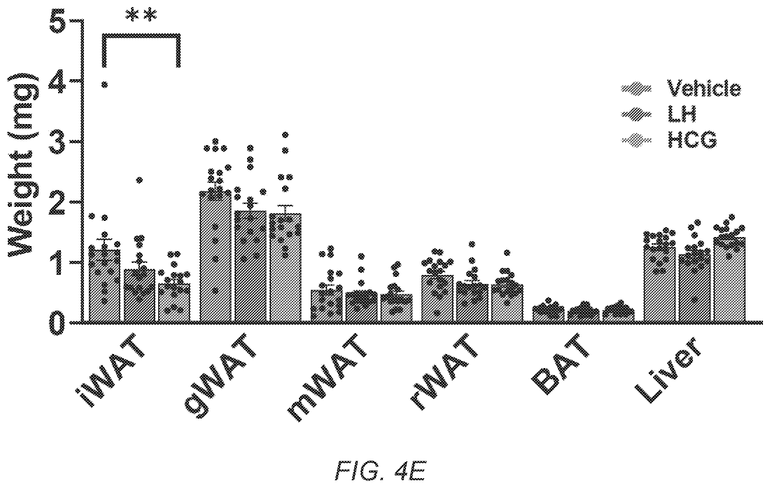

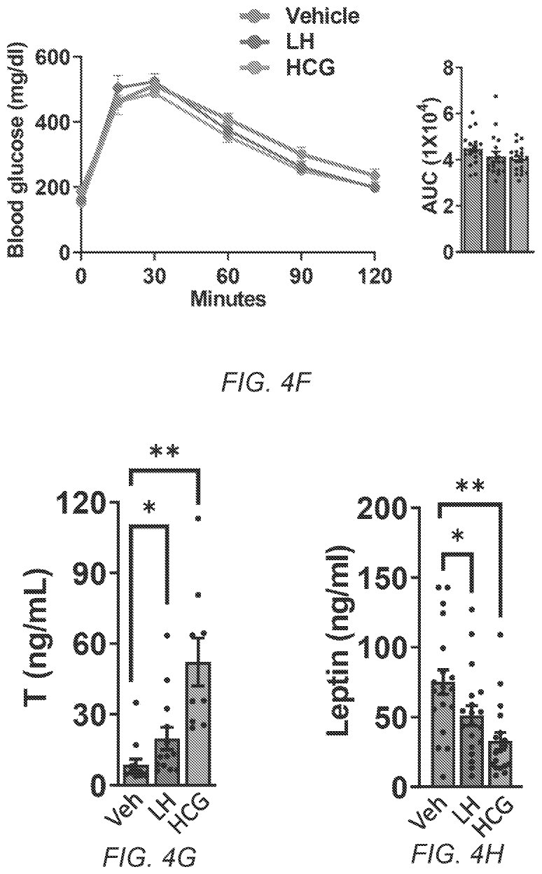

Fourteen-week-old C57BL/6 mice (20 mice/group) were treated with intraperitoneal injections of vehicle (200 μL saline), LH (250 ng/mouse) or hCG (250 ng/mouse), and fed on high-fat diet (TestDiet 0056833) for 6 weeks. As shown in FIGS. 4A and 4E, high-dose LH, injected twice-a-week into 14-week-old C57BL/6 male mice fed on high-fat diet, markedly reduced body fat mass. As shown in FIG. 4B, the same treatment increased lean mass, and as shown in FIG. 4C, the treatment decreased total mass. A shown in FIG. 4D, food intake was similar in all groups. As shown in FIG. 4A, hCG-treated mice displayed lower fat mass, and as shown in FIG. 4E, hCG-treated mice displayed lower iWAT weight. As shown in FIG. 4F, there was no difference in glucose tolerance among the three treatment groups. As shown in FIG. 4G, mice treated with LH or hCG displayed elevated testosterone. A shown in FIG. 4H, mice treated with hCG and LH had lower leptin levels.

### Example 5: Anti-Obesity Action of LH/hCG is Independent of Testosterone

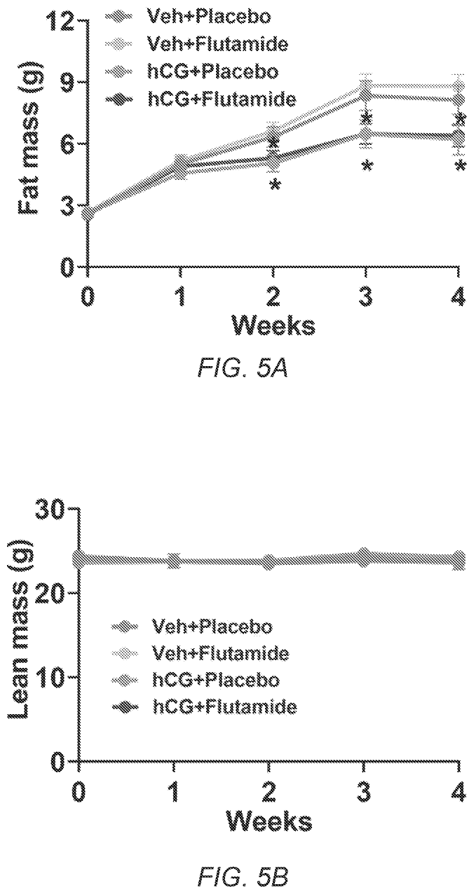

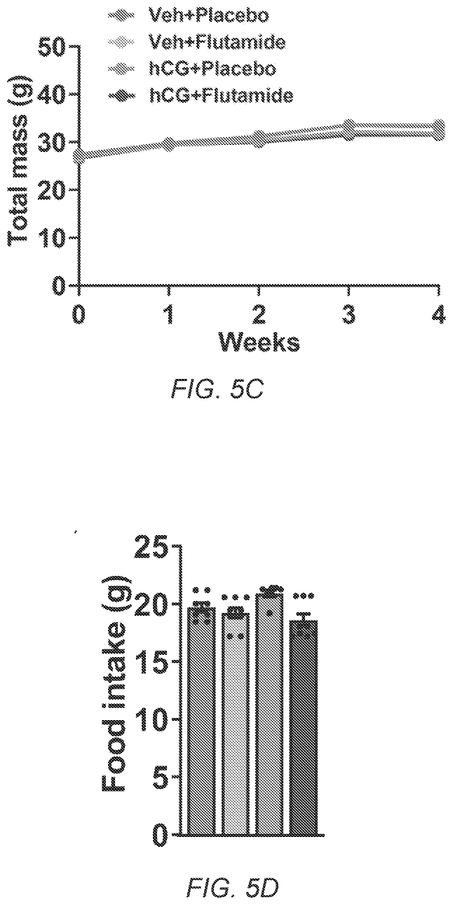

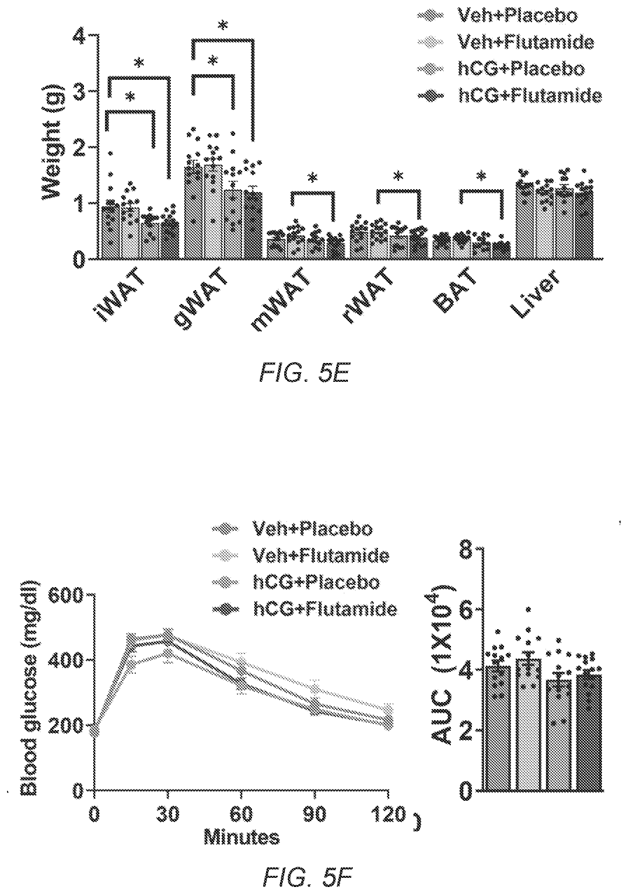

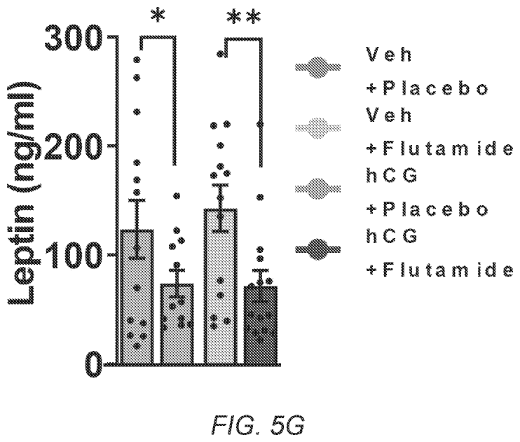

In order to evaluate the anti-obesity action of LH/hCG relative to testosterone levels, flutamide, an androgen receptor antagonist, was used to block the increase in testosterone after LH/hCG treatment in mice. Fourteen-week-old C57BL/6 mice (15 mice/group) underwent the following intraperitoneal injections—Group I: vehicle with placebo (placebo 60-day release pellets); Group II: vehicle with flutamide (7.5 mg 60-day release pellets); Group III: hCG (250 ng/mouse) with placebo; Group IV: hCG with flutamide. Mice were fed on high-fat diet for 6 weeks. As shown in FIG. 5A-5C, hCG reduced diet-induced fat mass (A), but not lean mass (B) or total mass (C), in the presence of flutamide, indicating a lack of a role of testosterone in the action of hCG in reducing body fat. A shown in FIG. 5E, mice treated with hCG had lower iWAT and gWAT weight in both flutamide and placebo groups. A shown in FIGS. 5D and 5F, food intake (D) and glucose tolerance (F) was similar in all groups. As shown in FIG. 5G, mice treated with hCG had lower leptin levels in both flutamide and placebo groups.

### Example 6: LHCGR Agonist ORG 43553 Reduces Body Fat

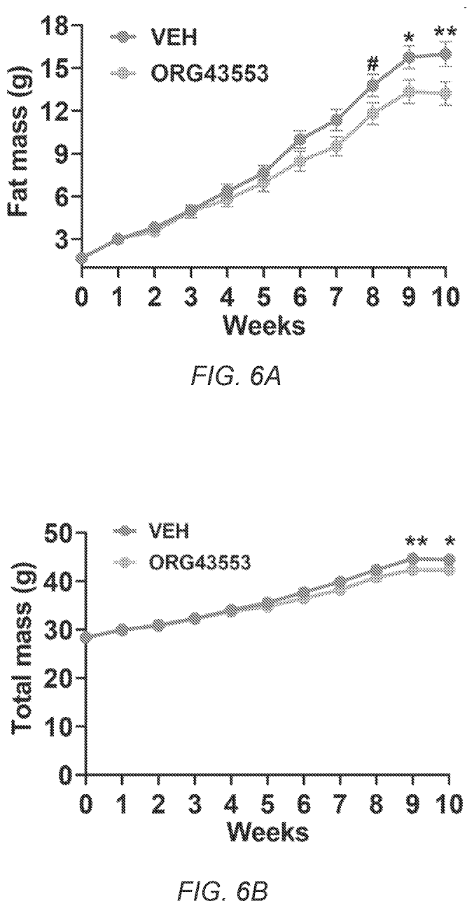

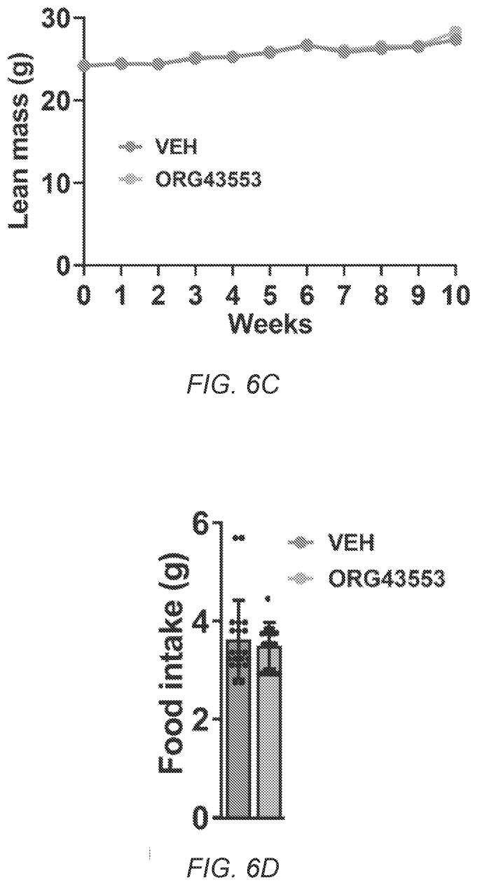

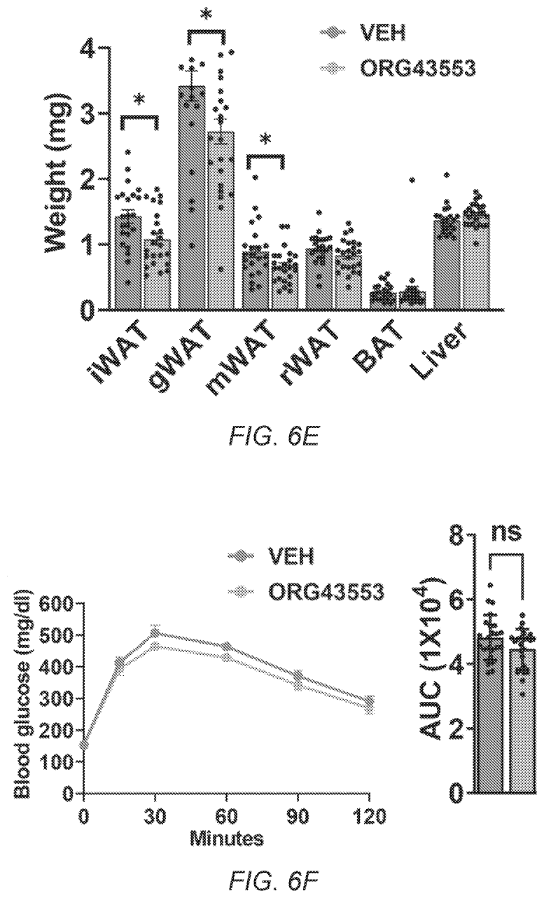

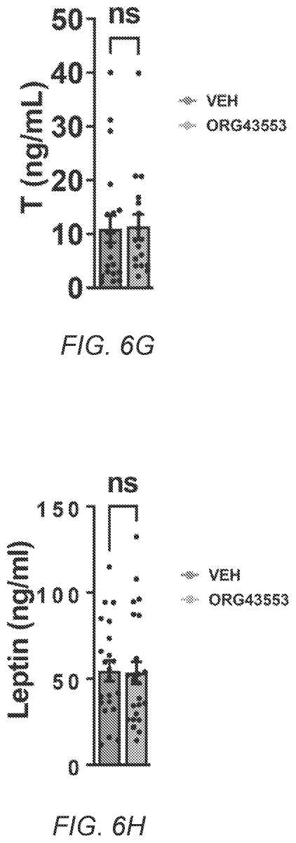

In order to evaluate the effect of treatment with LHCGR agonist ORG 43553 on body fat in mice, 14-week-old C57BL/6 mice (25 mice/group) were treated with subcutaneous injections of vehicle or ORG 43553 (17 mg/kg), and fed on high-fat diet for 9 weeks. As shown in FIGS. 6A-6C, ORG 43553 reduced diet-induced fat mass (A), total mass (B), but not lean mass (C) in mice. As shown in FIG. 6E, mice treated with ORG 43553 had lower iWAT, gWAT and mWAT weight. Food intake (FIG. 6D), glucose tolerance (FIG. 6F), testosterone (FIG. 6G), and leptin levels (FIG. 6H) were similar in all groups.

### Example 7: LHCGR Expression in Fat Tissue

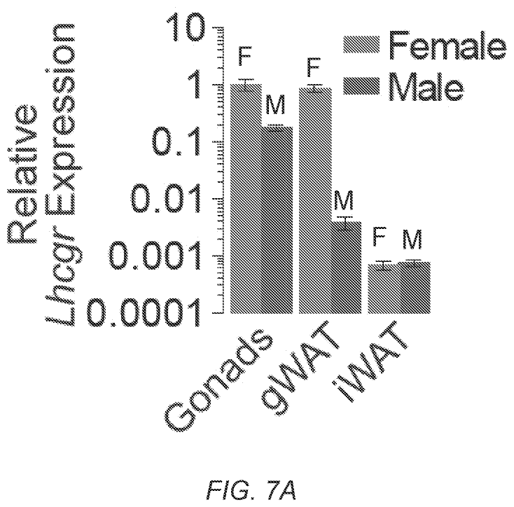

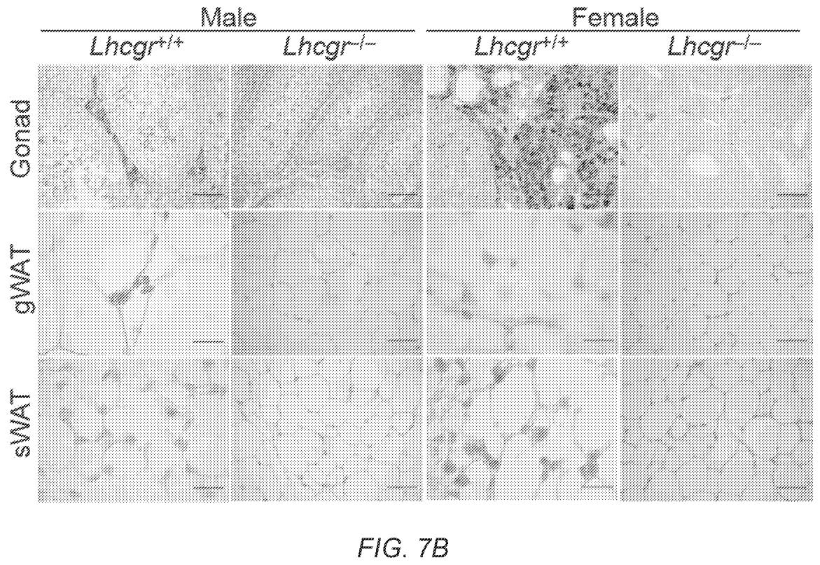

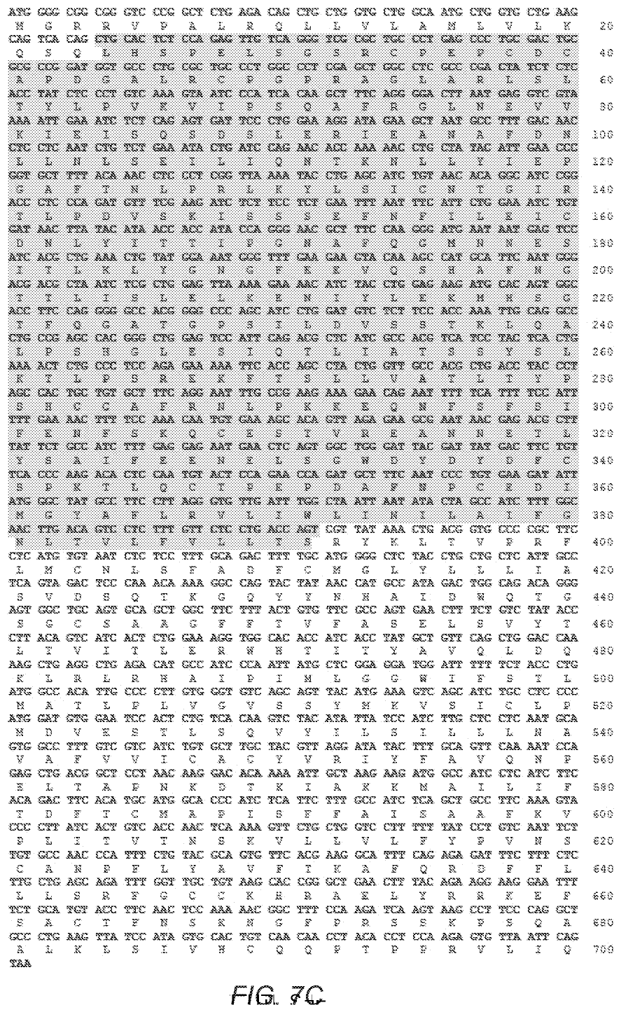

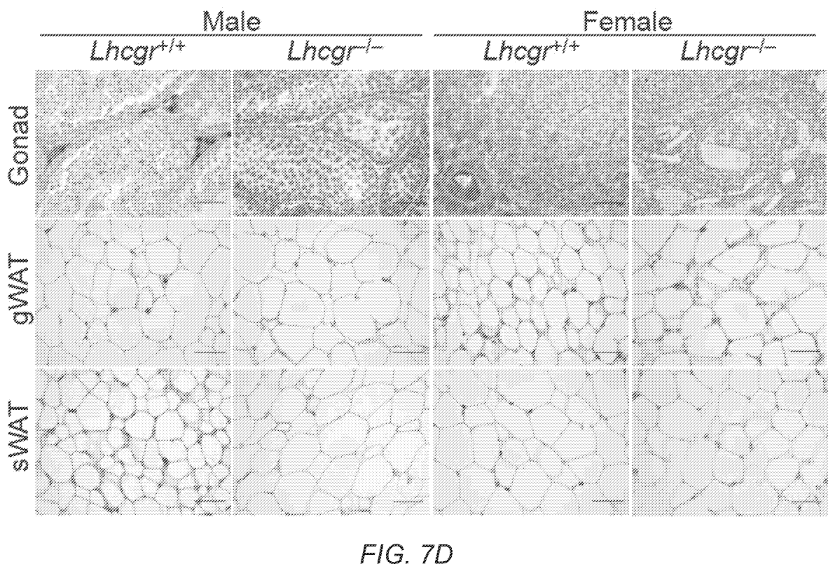

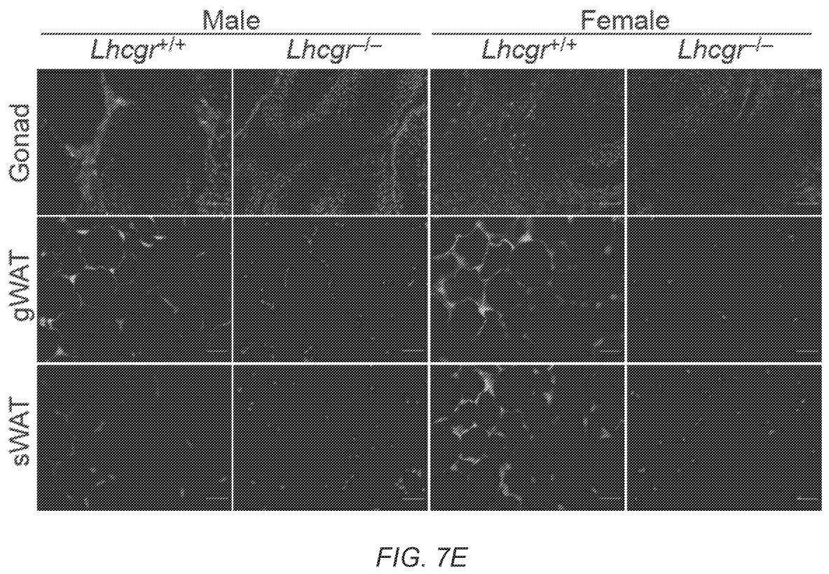

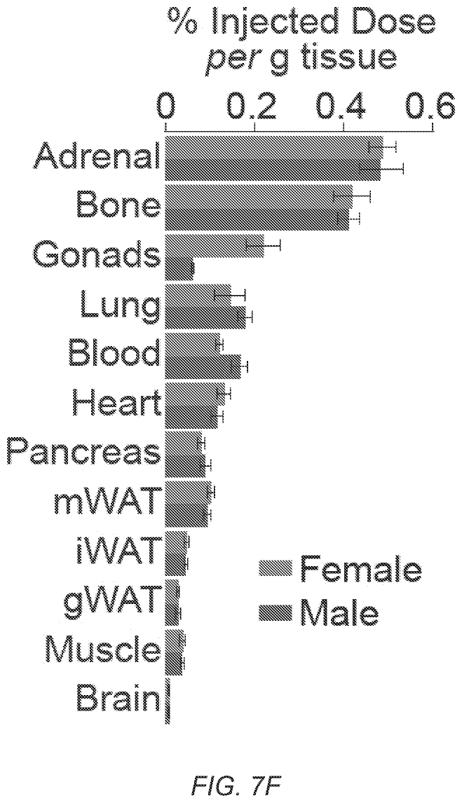

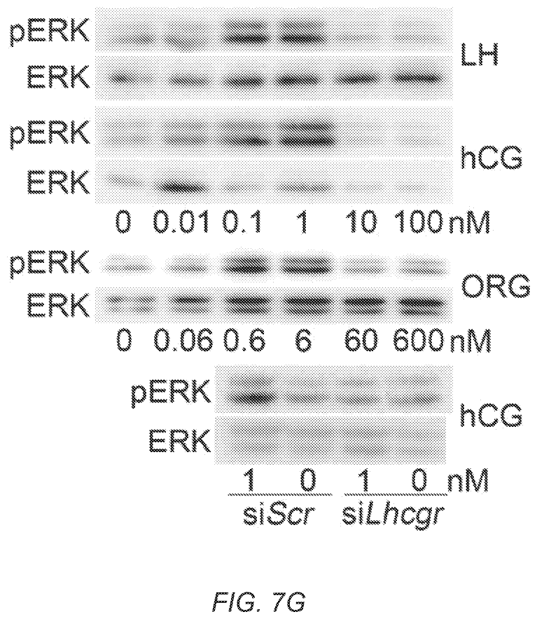

In order to characterize Lhcgr expression in adipose tissue, qPCR of Lhcgr transcripts, RNAscope™ in situ hybridization, and immunohistochemistry (IHC) were performed in mouse adipocytes. Relative expression of Lhcgr transcripts in white adipose tissue (WAT) depots in male and female C57BL/6 mice as measured by qPCR are shown in FIG. 7A. Expression in gonadal WAT (gWAT) in female mice approached that of the ovary itself (gonad). As shown in FIG. 7B, RNAscope™ in situ hybridization confirmed Lhcgr transcripts (dark dots) in gonads, gWAT and subcutaneous WAT (sWAT) in wild-type mice of both sexes, but not in Lhcgr^{-/-} mice. As shown in FIG. 7C, expression was validated by Sanger sequencing of the LHCGR ectodomain, which was identical to the ovarian receptor. As shown in FIG. 7D, immunohistochemistry indicated LHCGR expression in gWAT and sWAT.

The binding of labelled hCG was studied in vivo. As shown in FIG. 7E, intraperitoneal AlexaFluor-488-labeled hCG bound to gonadal and subcutaneous fatpads in wildtype mice, but not in LHCGR-null mice. Complementarily, ⁸⁹Zr-LH was injected intravenously, followed two hours later by sacrifice and perfusion with PBS before organ retrieval and γ counting. As shown in FIG. 7F, radioactivity was detected in mesenteric WAT (mWAT), inguinal WAT (iWAT) and gWAT, among other organs, including gonads. Finally, as shown in FIG. 7G, LH, hCG and ORG 43553 rapidly induced ERK1/2 phosphorylation in a concentration-dependent manner in differentiated 3T3.L1 cells, expectedly with no signal in siLhcgr cells. These results indicated that the adipose LHCGR was functional. Taken together, these results indicate adipose tissue LHCGRs as targets for the actions of LH.

### Example 8: LH Induces Weight Loss

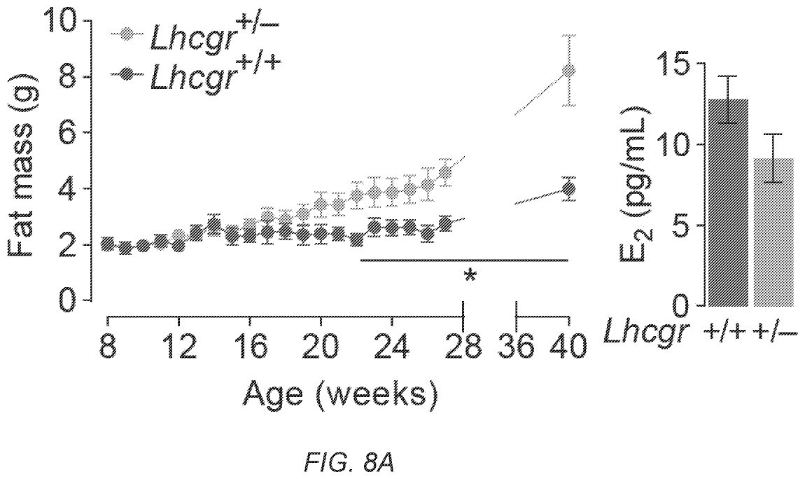

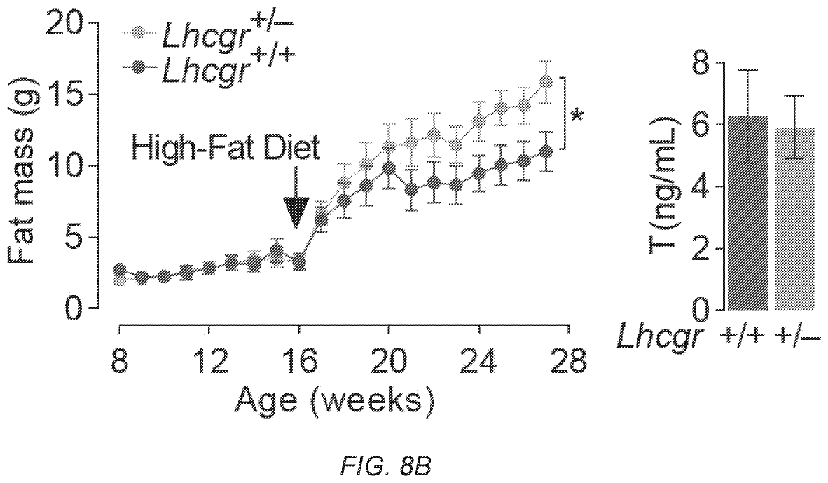

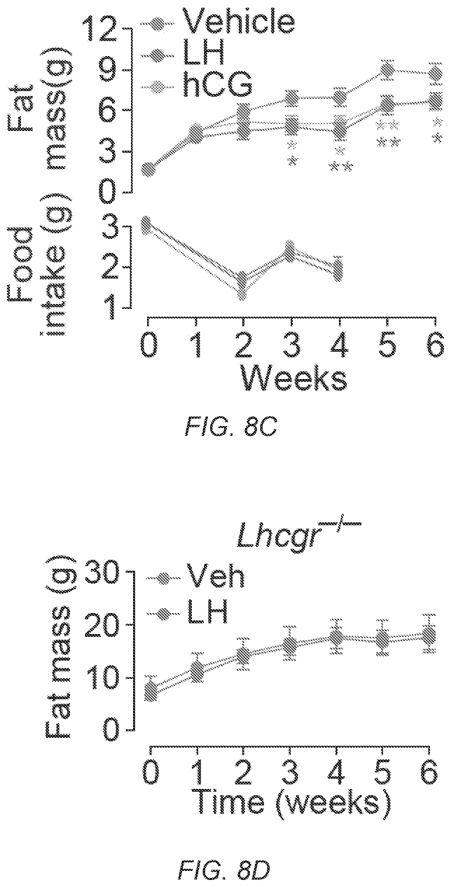

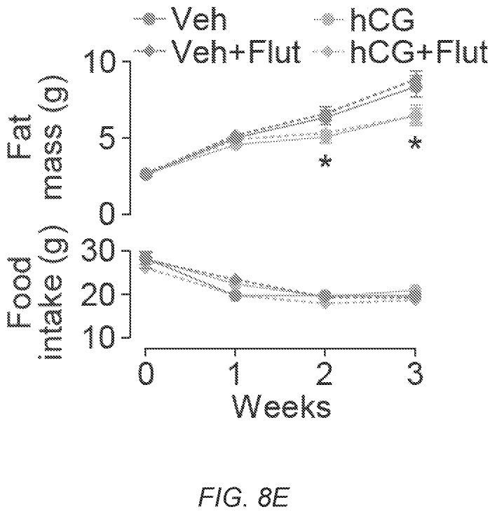

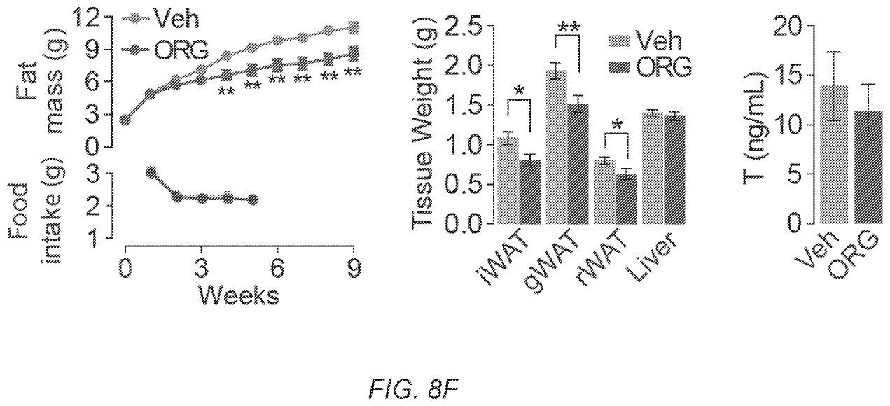

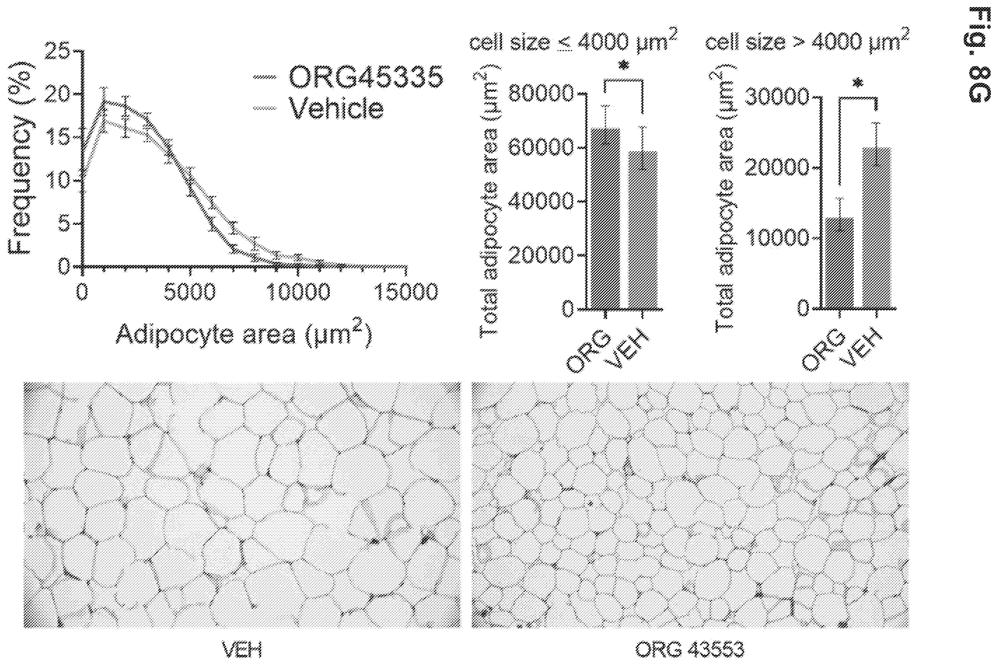

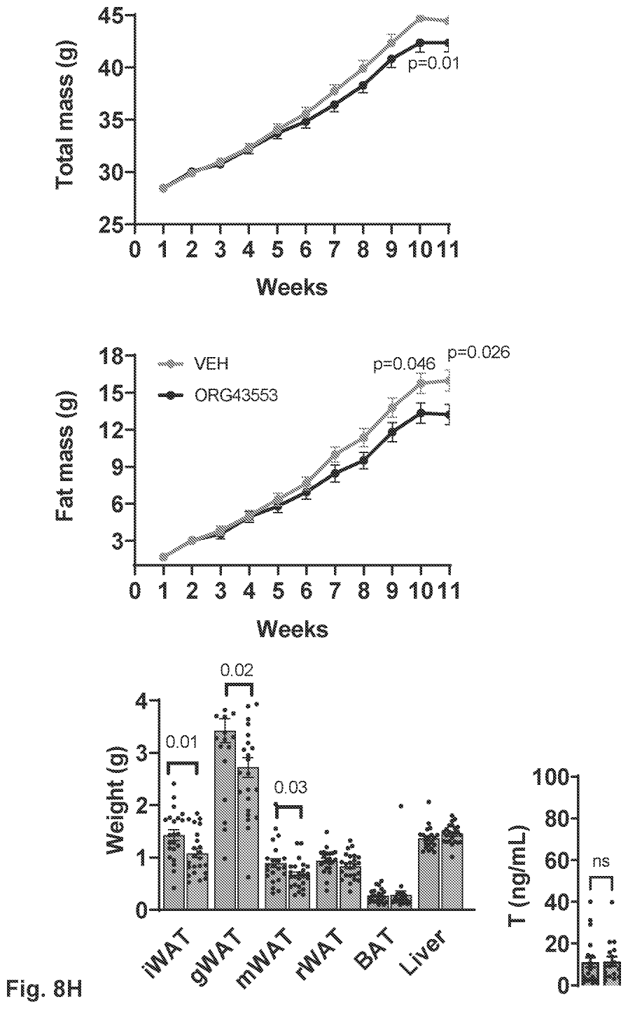

As shown in FIG. 8A, female Lhcgr^{+/-} mice on normal chow gained fat as measured by EchoMRI™-100, without a drop in serum estrogen. Likewise, as shown in FIG. 8B, male Lhcgr^{+/-} mice became obese on a high-fat diet (TestDiet0056833), importantly, without reduced serum testosterone. This gene-dosage effect of Lhcgr on adiposity suggests a dominant physiologic action of LH on body composition.

As shown in FIG. 8C, in gain-of-function studies, 14-week-old male C57BL/6mice (N=20/group) were fed on high-fat diet and injected intraperitoneally with LH (250 ng), hCG (250 ng) or vehicle (200 μL saline), twice-a-week, for 6 weeks. Both LH and hCG markedly reduced body fat accrual as measured by EchoMRI™_100 but triggered a rise in serum testosterone. As shown in FIG. 8D, that LH (250 ng) failed to inhibit fat accrual in Lhcgr^{-/-} mice testified to its action via the LHCGR—this experiment, however, did not isolate the effect of LH on adipose tissue LHCGRs from those on testosterone-producing Leydig cells. To address the confounding actions of high testosterone on bodyfat, the study was repeated in the presence of flutamide (7.5 mg, 60-day low release pellets). As shown in FIG. 8E, the hCG-induced lean phenotype persisted despite androgen receptor blockade by flutamide, indicating that the anti-adiposity actions of LH and hCG were largely independent of testosterone.

Next, the LHCGR agonist ORG 43553 was injected into male C57BL/6 mice (N=24/group) (17 mg/kg, subcutaneous, twice-a-week) for 9 weeks. As shown in FIG. 8F, a significant reduction in fat mass was observed, with reduced iWAT, gWAT and renal WAT (rWAT). Serum testosterone remained unchanged, indicating that the anti-adiposity effect of ORG 43553 was independent of testosterone. As shown in FIG. 8G, the distribution of adipocytes in iWAT indicates greater numbers of adipocytes with areas below 4000 μm² and fewer adipocytes with areas greater than 4000 μm² in mice treated with ORG 43553. As shown in FIG. 8H, injection of ORG 43553 into a replication cohort resulted in a significant reduction in fat mass and total mass, with reduced iWAT, gWAT and mWAT, independent of testosterone. These data indicate that LHCGR activation induces a lean phenotype. Further, male and female homozygotic Lhcgr^{-/-} mice displayed more pronounced obesity (data not shown), but this effect is likely to be confounded by accompanying hypogonadism, particularly at older ages.

### Example 9: LHCGR Agonism Inhibits Adipocyte Differentiation and Induces Thermogenesis

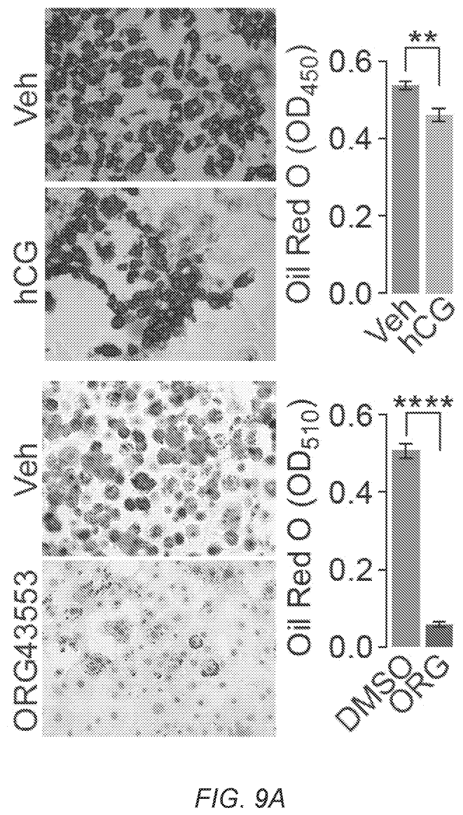

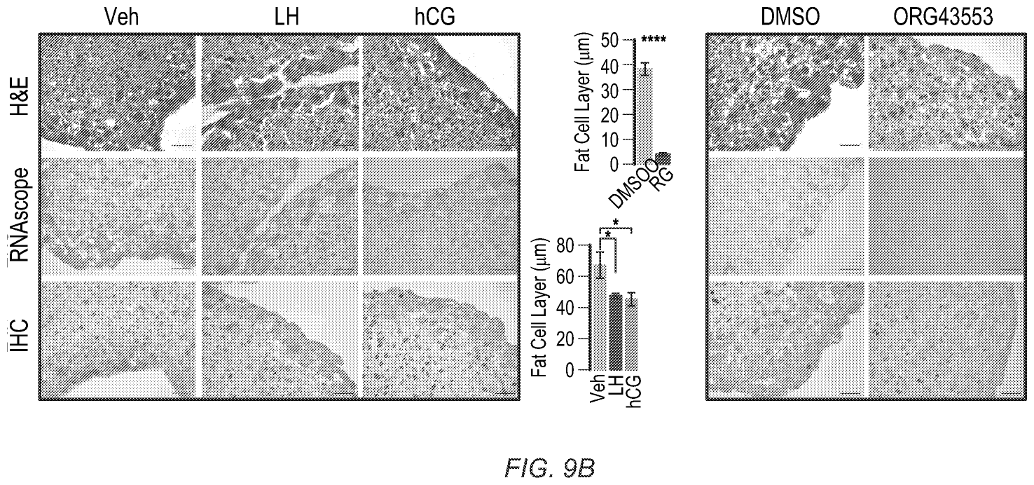

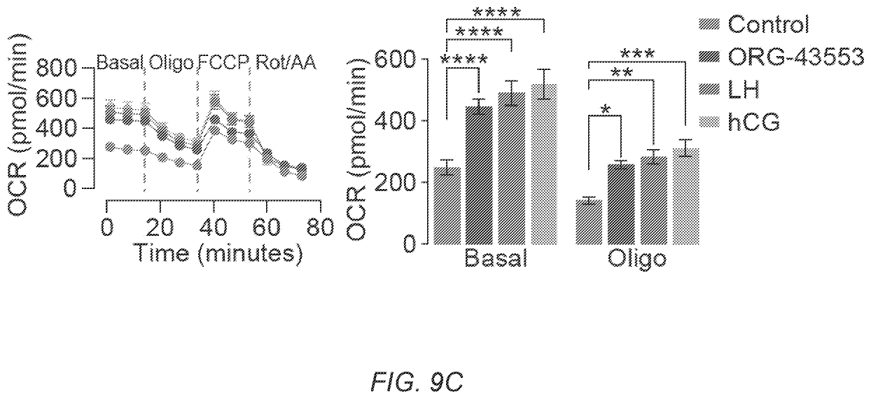

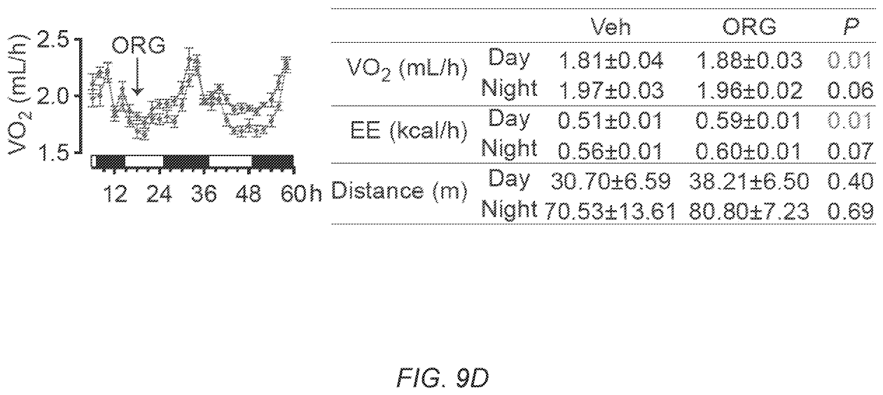

In order to study the mechanism of LHCGR activation on body fat, 3T3.L1 pre-adipocytes and adipose organoids were used. Oil Red 0 staining can be used to mark lipid-containing vacuoles and quantify cell lipid content. As shown in FIG. 9A, treatment of 3T3.L1 adipocytes with hCG (1 nM) or ORG 43553 (6 nM) for 12 hours resulted in reduced oil droplet accumulation (Oil Red O staining), indicating reduced lipid content. Organoids were derived from 3T3-L1 cells following 8 days culture on Nunclon Sphera dishes, and exposed to LH, hCG or ORG 43553 for 3 days. They were allowed to further differentiate for 7 days, after which paraffin sections were stained with H&E, and LHCGR expression was confirmed by immunostaining and RNAscope™. The thickness of the “differentiating cell layer” was measured with QuPath-0.2.3. As shown in the plots of FIG. 9B, organoids treated with LH, hCG or ORG 43553 displayed a substantial reduction in the differentiated layer compared to vehicle-treated organoids.

Next, 3T3-L1 adipocytes were treated with ORG 43553 in order to determine whether, in addition to reducing adipocyte differentiation, LHCGR agonism also enhanced thermogenesis. 3T3-L1 adipocytes were thus pretreated with ORG 43553 for 1 hour before measuring oxygen consumption rate (OCR) on a Seahorse Xf96 Analyzer. As shown in FIG. 9C, oligomycin (Oligo), FCCP and rotenone/antimycin-A were added at the indicated times. ORG 43553 increased OCR at baseline and upon oligomycin exposure, indicating mitochondria proton leak—a surrogate for thermogenesis. To confirm increased energy expenditure in vivo, mice (N=6/group) on high-fat diet were acclimated to metabolic cages for 1 day, before being injected subcutaneously with ORG 43553 (17 mg/kg, blue line) or vehicle and monitored for 48 hours. As shown in FIG. 9D, oxygen consumption (VO₂) and energy expenditure (EE) increased acutely in ORG 43553-treated mice, with no change in locomotor activity (distance). *P<0.05, **P<0.01, ***P<0.001, ****P<0.0001. These results indicate that the anti-obesity actions of LHCGR agonism arise from reduced adipocyte differentiation and increased thermogenesis.

### Example 10: Structure of ORG 43553 and Binding to the LHCGR

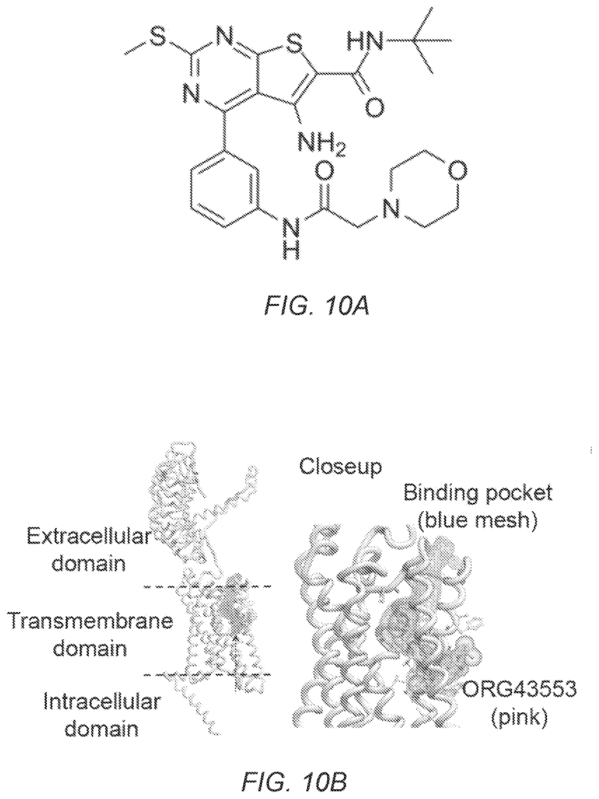

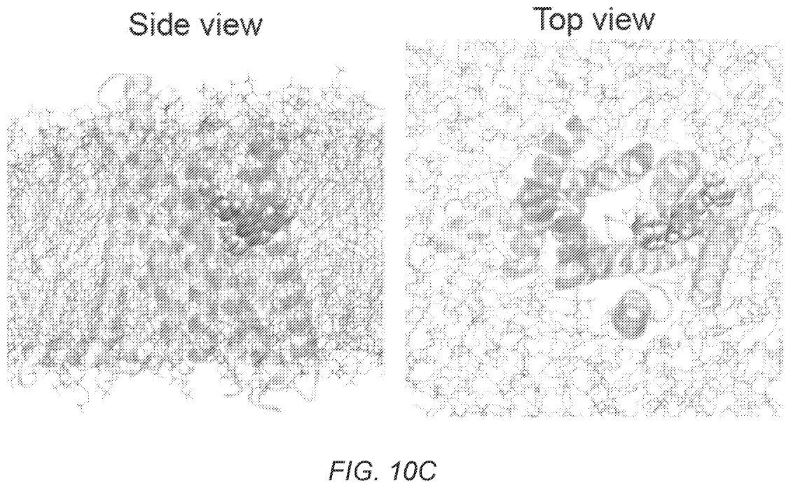

FIG. 10A shows the chemical structure of ORG 43553 [5-amino-2-methylsulfanyl-4-[3-(2-morpholin-4-yl-acetylamino)-phenyl]-thieno[2,3-d]pyrimidine-6-carboxylic acid tert-butylamide]. The binding of ORG 43553 with mouse LHCGR was modeled computationally. Unlike LH which binds to the ectodomain, ORG 43553 binds to the transmembrane domain (TMD) (FIG. 10B). FIG. 10C shows side (left) and top (right) views of ORG 43553 in complex with LHCGR on 280 nanosecond molecular dynamics.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating obesity in a subject in need thereof, comprising administering to the subject in need thereof a thieno[2,3-d]pyrimidine derivative according to general formula I, 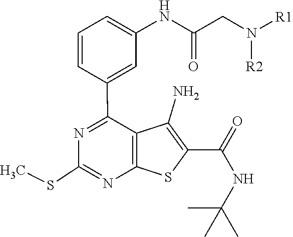
or a pharmaceutically acceptable salt thereof, wherein R1 and R2 together with the nitrogen atom to which they are bonded form a ring having 2-6 carbon atoms, optionally containing one or more heteroatoms selected from N, O and/or S.

2. The method of claim 1, wherein the thieno[2,3-d]pyrimidine derivative is a compound selected from the group consisting of tert-butyl 5-amino-2-methylthio-4-(3-(2-(azetidin-1-yl)-acetamido)-phenyl)-thieno[2,3-d]pyrimidine-6-carboxamide; tert-butyl 5-amino-2-methylthio-4-(3-(2-(morpholin-4-yl)-acetamido)-phenyl)-thieno [2,3-d]pyrimidine-6-carboxamide; tert-butyl 5-amino-2-methylthio-4-(3-(2-(thiomorpholin-4-yl)-acetamido)-phenyl)-thieno[2,3-d]pyrimidine-6-carboxamide; tert-butyl 5-amino-2-methylthio-4-(3-(2-(piperidin-1-yl)-acetamido)-phenyl)-thieno[2,3-d]pyrimidine-6-carboxamide; tert-butyl 5-amino-2-methylthio-4-(3-(2-(pyrrolidin-1-yl)-acetamido)-phenyl)-thieno[2,3-d]pyrimidine-6-carboxamide or tert-butyl 5-amino-2-methylthio-4-(3-(2-(piperazin-1-yl)-acetamido)-phenyl)-thieno[2,3-d]pyrimidine-6-carboxamide.

3. The method of claim 1 or claim 2, wherein the thieno[2,3-d]pyrimidine derivative is ORG 43533.

4. The method of any one of claims 1-3, wherein the thieno[2,3-d]pyrimidine derivative is administered at a dosage of between 1 and 100 mg/kg.

5. The method of claim 4, wherein the thieno[2,3-d]pyrimidine derivative is administered at a dosage of between 1 and 50 mg/kg.

6. The method of claim 5, wherein the thieno[2,3-d]pyrimidine derivative is administered at a dosage of between 1 and 30 mg/kg.

7. The method of claim 6, wherein the thieno[2,3-d]pyrimidine derivative is administered at a dosage of between 1 and 20 mg/kg.

8. The method of claim 7, wherein the thieno[2,3-d]pyrimidine derivative is administered at a dosage of between 10 and 20 mg/kg.

9. The method of claim 8, wherein the thieno[2,3-d]pyrimidine derivative is administered at a dosage of 17 mg/kg.

10. The method of any one of claims 1-9 wherein the thieno[2,3-d]pyrimidine derivative is administered daily.

11. The method of any one of claims 1-9 wherein the thieno[2,3-d]pyrimidine derivative is administered weekly.

12. The method of any one of claims 1-11 wherein the thieno[2,3-d]pyrimidine derivative is administered for at least 26 weeks.

13. The method of any one of claims 1-12 wherein the thieno[2,3-d]pyrimidine derivative is provided as a pharmaceutical composition including a pharmaceutically acceptable carrier.

14. The method of any one of claims 1-13, further comprising administering to the subject in need thereof a luteinizing hormone (LH).

15. A method of treating diet-induced obesity in a subject in need thereof, comprising administering to the adipose tissue of a subject in need thereof a thieno[2,3-d]pyrimidine derivative according to general formula I, 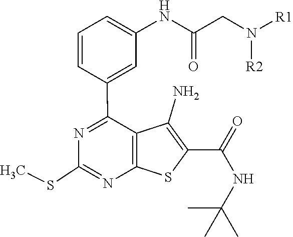
or a pharmaceutically acceptable salt thereof, wherein R1 and R2 together with the nitrogen atom to which they are bonded form a ring having 2-6 carbon atoms, optionally containing one or more heteroatoms selected from N, O and/or S.

16. The method of claim 15, wherein the thieno[2,3-d]pyrimidine derivative is a compound selected from the group consisting of tert-butyl 5-amino-2-methylthio-4-(3-(2-(azetidin-1-yl)-acetamido)-phenyl)-thieno[2,3-d]pyrimidine-6-carboxamide; tert-butyl 5-amino-2-methylthio-4-(3-(2-(morpholin-4-yl)-acetamido)-phenyl)-thieno [2,3-d]pyrimidine-6-carboxamide; tert-butyl 5-amino-2-methylthio-4-(3-(2-(thiomorpholin-4-yl)-acetamido)-phenyl)-thieno[2,3-d]pyrimidine-6-carboxamide; tert-butyl 5-amino-2-methylthio-4-(3-(2-(piperidin-1-yl)-acetamido)-phenyl)-thieno[2,3-d]pyrimidine-6-carboxamide; tert-butyl 5-amino-2-methylthio-4-(3-(2-(pyrrolidin-1-yl)-acetamido)-phenyl)-thieno[2,3-d]pyrimidine-6-carboxamide or tert-butyl 5-amino-2-methylthio-4-(3-(2-(piperazin-1-yl)-acetamido)-phenyl)-thieno[2,3-d]pyrimidine-6-carboxamide.

17. The method of claim 15 or 16, wherein the thieno[2,3-d]pyrimidine derivative is ORG 43902.

18. The method of any one of claims 15-17, wherein the thieno[2,3-d]pyrimidine derivative is administered at a dosage of between 1 and 100 mg/kg.

19. The method of claim 18, wherein the thieno[2,3-d]pyrimidine derivative is administered at a dosage of between 1 and 50 mg/kg.

20. The method of claim 19, wherein the thieno[2,3-d]pyrimidine derivative is administered at a dosage of between 1 and 30 mg/kg.

21. The method of claim 20, wherein the thieno[2,3-d]pyrimidine derivative is administered at a dosage of between 1 and 20 mg/kg.

22. The method of claim 21, wherein the thieno[2,3-d]pyrimidine derivative is administered at a dosage of between 10 and 20 mg/kg.

23. The method of claim 22, wherein the thieno[2,3-d]pyrimidine derivative is administered at a dosage of 17 mg/kg.

24. The method of any one of claims 15-23 wherein the thieno[2,3-d]pyrimidine derivative is administered daily.

25. The method of any one of claims 15-23 wherein the thieno[2,3-d]pyrimidine derivative is administered weekly.

26. The method of any one of claims 15-25 wherein the thieno[2,3-d]pyrimidine derivative agonist is administered for at least 26 weeks.

27. The method of any one of claims 15-26 wherein the thieno[2,3-d]pyrimidine derivative is provided as a pharmaceutical composition including a pharmaceutically acceptable carrier.

28. The method of any one of claims 15-27, further comprising administering to the subject in need thereof a luteinizing hormone (LH).

29. A method of treating obesity in a subject in need thereof, comprising administering to a subject in need thereof a combination of ORG 43553 and ORG 43902.


SEQUENCE LISTING (5 sequences)

SEQ ID NO: 1 - protein, 699 aa, Homo sapiens
MKQRFSALQLLKLLLLLQPPLPRALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPRLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPSHCCAFRNLPTKEQNFSHSISENFSKQCESTVRKVNNKTLYSSMLAESELSGWDYEYGFCLPKTPRCAPEPDAFNPCEDIMGYDFLRVLIWLINILAIMGNMTVLFVLLTSRYKLTVPRFLMCNLSFADFCMGLYLLLIASVDSQTKGQYYNHAIDWQTGSGCSTAGFFTVFASELSVYTLTVITLERWHTITYAIHLDQKLRLRHAILIMLGGWLFSSLIAMLPLVGVSNYMKVSICFPMDVETTLSQVYILTILILNVVAFFIICACYIKIYFAVRNPELMATNKDTKIAKKMAILIFTDFTCMAPISFFAISAAFKVPLITVTNSKVLLVLFYPINSCANPFLYAIFTKTFQRDFFLLLSKFGCCKRRAELYRRKDFSAYTSNCKNGFTGSNKPSQSTLKLSTLHCQGTALLDKTRYTEC

SEQ ID NO: 2 - DNA, 2103 bp, Mus musculus
  source 1..2103: mol_type = genomic DNA
atggggcggcgggtcccggctctgagacagctgctggtgctggcaatgctggtgctgaagcagtcacagctgcactctccagagttgtcagggtcgcgctgccctgagccctgcgactgcgcgccggatggtgccctgcgctgccctggccctcgagctggcctcgcccgactatctctcacctatctccctgtcaaagtaatcccatcacaagctttcaggggacttaatgaggtcgtaaaaattgaaatctctcagagtgattccctggaaaggatagaagctaatgcctttgacaacctcctcaatctgtctgaaatactgatccagaacaccaaaaacctgctatacattgaacccggtgcttttacaaacctccctcggttaaaatacctgagcatctgtaacacaggcatccggaccctcccagatgtttcgaagatctcttcctctgaatttaatttcattctggaaatctgtgataacttatacataaccaccataccagggaacgctttccaagggatgaataatgagtccatcacgctgaaactgtatggaaatgggtttgaagaagtacaaagccatgcattcaatgggacgacgctaatctcgctggagttaaaagaaaacatctacctggagaagatgcacagtggcaccttccagggggccacggggcccagcatcctggatgtctcttccaccaaattgcaggccctgccgagccacgggctggagtccattcagacgctcatcgccacgtcatcctactcactgaaaactctgccctccagagaaaaattcaccagcctactggttgccacgctgacctaccctagccactgctgtgctttcaggaatttgccgaagaaagaacagaatttttcattttccatttttgaaaacttttccaaacaatgtgaaagcacagttagagaagcgaataacgagacgctttattctgccatctttgaggagaatgaactcagtggctgggattacgattatgacttctgttcacccaagacactccaatgtactccagaaccagatgctttcaatccctgtgaagatattatgggctatgccttccttagggtgttgatttggctaattaatatactagccatctttggcaacttgacagtcctctttgttctcctgaccagtcgttataaactgacggtgccccgcttcctcatgtgtaatctctcctttgcagacttttgcatggggctctacctgctgctcattgcctcagtagactcccaaacaaaaggccagtactataaccatgccatagactggcagacagggagtggctgcagtgcagctggcttctttactgtgttcgccagtgaactttctgtctatacccttacagtcatcactctggaaaggtggcacaccatcacctatgctgttcagctggaccaaaagctgaggctgagacatgccatcccaattatgctcggaggatggattttttctaccctgatggccacattgccccttgtgggtgtcagcagttacatgaaagtcagcatctgcctccccatggatgtggaatccactctgtcacaagtctacatattatccatcttgctcctcaatgcagtggcctttgtcgtcatctgtgcttgctacgttaggatatactttgcagttcaaaatccagagctgacggctcctaacaaggacacaaaaattgctaagaagatggccatcctcatcttcacagacttcacatgcatggcacccatctcattctttgccatctcagctgccttcaaagtaccccttatcactgtcaccaactcaaaagttctgctggtccttttttatcctgtcaattcttgtgccaacccatttctgtacgcagtgttcacgaaggcatttcagagagatttctttctcttgctgagcagatttggttgctgtaagcaccgggctgaactttacagaaggaaggaattttctgcatgtaccttcaactccaaaaacggctttccaagatcaagtaagccttcccaggctgccctgaagttatccatagtgcactgtcaacaacctacacctccaagagtgttaattcagtaa

SEQ ID NO: 3 - protein, 700 aa, Mus musculus
MGRRVPALRQLLVLAMLVLKQSQLHSPELSGSRCPEPCDCAPDGALRCPGPRAGLARLSLTYLPVKVIPSQAFRGLNEVVKIEISQSDSLERIEANAFDNLLNLSEILIQNTKNLLYIEPGAFTNLPRLKYLSICNTGIRTLPDVSKISSSEFNFILEICDNLYITTIPGNAFQGMNNESITLKLYGNGFEEVQSHAFNGTTLISLELKENIYLEKMHSGTFQGATGPSILDVSSTKLQALPSHGLESIQTLIATSSYSLKTLPSREKFTSLLVATLTYPSHCCAFRNLPKKEQNFSFSIFENFSKQCESTVREANNETLYSAIFEENELSGWDYDYDFCSPKTLQCTPEPDAFNPCEDIMGYAFLRVLIWLINILAIFGNLTVLFVLLTSRYKLTVPRFLMCNLSFADFCMGLYLLLIASVDSQTKGQYYNHAIDWQTGSGCSAAGFFTVFASELSVYTLTVITLERWHTITYAVQLDQKLRLRHAIPIMLGGWIFSTLMATLPLVGVSSYMKVSICLPMDVESTLSQVYILSILLLNAVAFVVICACYVRIYFAVQNPELTAPNKDTKIAKKMAILIFTDFTCMAPISFFAISAAFKVPLITVTNSKVLLVLFYPVNSCANPFLYAVFTKAFQRDFFLLLSRFGCCKHRAELYRRKEFSACTFNSKNGFPRSSKPSQAALKLSIVHCQQPTPPRVLIQ

SEQ ID NO: 4 - DNA, 1074 bp, Mus musculus
  source 1..1074: mol_type = genomic DNA
ccagagttgtcagggtcgcgctgccctgagccctgcgactgcgcgccggatggtgccctgcgcttccctggccctcgagctggcctcgcccgactatctctcacctatctccctgtcaaagtaatcccatcacaagctttcaggggacttaatgaggtcgtaaaaattgaaatctctcagagtgattccctggaaaggatagaagctaatgcctttgacaacctcctcaatctgtctgaaatactgatccagaacaccaaaaacctgctatacattgaacccggtgcttttacaaacctccctcggttaaaatacctgagcatctgtaacacaggcatccggaccctcccagatgtttcgaagatctcttcctctgaatttaatttcattctggaaatctgtgataacttatacataaccaccataccagggaacgctttccaagggatgaataatgagtccatcacgctgaaactgtatggaaatgggtttgaagaagtacaaagccatgcattcaatgggacgacgctaatctcgctggagttaaaagaaaacatctacctggagaagatgcacagtggcaccttccagggggccacggggcccagcatcctggatgtctccaccaaattgcaggccctgccgagccacgggctggagtccattcagacgctcatcgccacgtcatcctactcactgaaaactctgccctccagagaaaaattcaccagcctactggttgccacgctgacctaccctagccactgctgtgctttcaggaatttgccgaagaaagaacagaatttttcattttccatttttgaaaacttttccaaacaatgtgaaagcacagttagagaagcgaataacgagacgctttattctgccatctttgaggagaatgaactcagtggctgggattacgattatgacttctgttcacccaagacactccaatgtactccagaaccagatgctttcaatccctgtgaagatattatgggctatgccttccttagggtgttgatttggctaattaatatactagccatctttggcaacttgacagtcctc

SEQ ID NO: 5 - protein, 357 aa, Mus musculus
ELSGSRCPEPCDCAPDGALRFPGPRAGLARLSLTYLPVKVIPSQAFRGLNEVVKIEISQSDSLERIEANAFDNLLNLSEILIQNTKNLLYIEPGAFTNLPRLKYLSICNTGIRTLPDVSKISSSEFNFILEICDNLYITTIPGNAFQGMNNESITLKLYGNGFEEVQSHAFNGTTLISLELKENIYLEKMHSGTFQGATGPSILDVSTKLQALPSHGLESIQTLIATSSYSLKTLPSREKFTSLLVATLTYPSHCCAFRNLPKKEQNFSFSIFENFSKQCESTVREANNETLYSAIFEENELSGWDYDYDFCSPKTLQCTPEPDAFNPCEDIMGYAFLRVLIWLINILAIFGNLTVL